(19) United States
(12) Patent Application Publication
(10) Pub. No.: US 2007/0154393 A1
(43) Pub. Date: Jul. 5, 2007

(54) Bioassay for volatile low molecular weight insecticides and methods of use

(76) Inventors: Michael E. Scharf, Gainesville, FL (US); Sam N. Nguyen, Gainesville, FL (US); Cheol Song, Gainesville, FL (US); Phillip G. Koehler, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/606,823

(22) Filed: Nov. 29, 2006

Related U.S. Application Data
(60) Provisional application No. 60/740,452, filed on Nov. 29, 2005.

(51) Int. Cl. A61K 49/00; A01N 25/00

(52) U.S. Cl. 424/9.2; 424/405

(57) ABSTRACT

The subject invention pertains to materials and methods for screening of volatile insecticides for activity against pests, such as those that pose a threat to public health (e.g., dipterans such as flies and mosquitoes). One aspect of the invention pertains to an apparatus and bioassay for screening volatile compounds for activity against pests. The subject invention also concerns methods of using volatile compounds as insecticides against pests that pose a threat to public health, such as flies and mosquitoes. The compounds used in the present methods can be formulated for use as an insecticide. The subject invention also concerns volatile compounds formulated for use as insecticides against pests that pose a threat to public health, such as flies and mosquitoes.

### CROSS-REFERENCE TO RELATED APPLICATION

This application claims the benefit of U.S. Provisional Application Ser. No. 60/740,452, filed Nov. 29, 2005, which is hereby incorporated by reference in its entirety, including all figures and tables.

This invention was made with government support under U.S. Space and Missile Defense Command grant number W9113M-05-1-0009. The government has certain rights in the invention.

### BACKGROUND OF THE INVENTION

Volatile insecticides have long had applications in the protection of agricultural crops, stored products and commodities, as well as in the control and management of structural pests (Brown, 1951; Mallis, 1954). The most effective of these volatile compounds, also know as fumigants, include phosphine and methyl bromide. However, phosphine has slow action and methyl bromide, while highly effective, is being phased out because of its role in ozone depletion (Bell, 2000; Caddick 2004). An additional drawback is that high levels of insect resistance to phosphine have developed in some areas as a result of its widespread over-use (Caddick 2004). Alkyl-ester fumigants such as ethyl formate and ethyl acetate have long been known as effective alternatives to more traditional fumigants (Brown, 1951). In particular, ethyl formate has proven very effective against coleopteran stored product pests (Ferguson et al., 1948; Haritos et al., 2003); and it is now commercially registered in Australia for pest control uses in dried fruits (Caddick 2004). Thus, the efficacy of some passively volatile fumigant materials have been demonstrated against stored product pests. However, only a narrow sampling of other available volatile compounds have been tested to date (e.g., Ferguson et al., 1948; Haritos et al., 2003; Park et al., 2005). Furthermore, virtually nothing is known regarding the efficacy of any volatile insecticides against other insect groups, particularly dipteran pests of medical importance.

There remains a need in the art for an assay for screening for volatile compounds that are effective against insects, such as flies and mosquitoes, and for compounds with insecticidal activity against these pests.

### BRIEF SUMMARY OF THE INVENTION

The subject invention concerns materials and methods for screening of volatile compounds for activity against insects and other pests, such as those that pose a threat to public health (e.g., dipterans such as flies and mosquitoes). Information on potential efficacy of volatile low molecular weight compounds against such pests can be obtained using the bioassay of the present invention. One aspect of the invention pertains to a bioassay and apparatus for screening volatile compounds for activity against pests. The subject invention also concerns volatile compounds that have been identified using the present invention. The compounds can be formulated for use as pesticides. The subject invention also concerns methods of using volatile compounds as pesticides against pests.

### BRIEF DESCRIPTION OF THE DRAWINGS

FIG. 1 shows a bioassay apparatus according to the present invention.

FIGS. 2A-2F show concentration-mortality plots for thirty compounds from six categories that include: heterobicyclics, formates, acetates, propionates, butyrates, and valerates. Each data point represents average % mortality determined from five replicates. All data shown were analyzed by probit analysis and used to generate data presented in Table 1.

FIGS. 3A-3C show regression analyses of LC₅₀ versus the physical properties of each of the thirty test compounds. Physical properties of the compounds that were evaluated include (FIG. 3A) molecular weight, (FIG. 3B) density and (FIG. 3C) boiling point. Line equations, correlation coefficients (r²) and p-values were determined by regression analysis. The six most effective insecticides are circled and abbreviated as follows: MF (menthofuran), THIO (benzothiophene), BF (butyl formate), HEXF (hexyl formate), HEPF (heptyl formate) and COUM (coumaran).

FIG. 4 shows toxicity of volatile low molecular weight insecticides to insecticide-susceptible Drosophila (Canton-S strain) using a volatility bioassay. Overall, 30 insecticidal compounds were tested. Vertical arrows ( [symbol] ) indicate the insecticidal compounds tested in the current study; solid arrows denote the seven top candidate insecticides, while open arrows denote reference compounds used as positive controls and for structure-activity comparisons. Results are summarized from Scharf et al., (2006).

FIG. 5 shows toxicity of experimental volatile insecticides, and two volatile “positive control” insecticides (DDVP and MITC) to the insecticide-susceptible Canton-S strain and the enzymatically-resistant Hikone-R strain. Black and white bars, respectively, represent Canton-S and Hikone-R (Canton-S normalized to 1.0). The Y-axis represents LC₅₀ ratios of Hikone÷Canton. Ratios >1 indicate resistance by Hikone-R, while ratios <1 indicate enhanced susceptibility, i.e., negative cross resistance (Pittendrigh and Gaffney, 2001). Asterisks (*) denote ratios that are significant at p<0.05 based on the method of Robertson and Preisler (1992).

FIGS. 6A-6D show the effects of synergists that inhibit detoxification enzymes on the toxicity of volatile insecticides to the insecticide-susceptible Canton-S (FIGS. 6A and 6B) and enzymatically-resistant Hikone-R (C, D) strains. The two inhibitors tested were the cytochrome P450 inhibitor PBO (FIGS. 6A and 6C) and the esterase inhibitor DEF (FIGS. 6B and 6D). Black and gray bars, respectively, represent the Canton-S and Hikone-R strains. The Y-axis represents synergist ratios of LC50s with synergist treatment÷LC₅₀s without synergist treatment. Ratios <1 indicate increases in toxicity after enzyme inhibition, while ratios >1 indicate reduced toxicity after inhibition. Asterisks (*) denote ratios that are significant at p<0.05 based on the method of Robertson and Preisler (1992).

FIGS. 7A-7B show toxicity of experimental volatile insecticides to insecticide susceptible (Canton-S) and the neurologically-resistant Rd1 (FIG. 7A) and para-ts1 (FIG. 7B) strains. Formic acid, the hydrolysis product and presumed toxic metabolite of the formate ester insecticides (Haritos and Dojchinov 2003) was also included in these bioassays. Black and gray bars, respectively, represent the susceptible and resistant strains (Canton-S normalized to 1.0). The Y-axis represents LC₅₀ ratios of each resistant strain÷Canton-S. Ratios >1 indicate resistance by neurological mutant strains, while ratios <1 indicate enhanced susceptibility, or “negative cross resistance” (Pittendrigh and Gaffney, 2001). Asterisks (*) denote ratios that are significant at p<0.05 based on the method of Robertson and Preisler (1992)

### DETAILED DISCLOSURE OF THE INVENTION

The subject invention concerns materials and methods for screening of passively volatile compounds for killing activity against insect pests, and in particular, dipterans that pose a threat to agriculture and/or public health, such as flies and mosquitoes.

One aspect of the invention pertains to a bioassay for screening volatile compounds for activity to kill or knockdown pests, such as insect pests. As used herein, the term “knockdown” refers to a condition wherein a pest (e.g., an insect) does not function in a normal manner (e.g., where a flying insect cannot fly or a non-flying insect cannot perform normal locomotion) even though the pest is still alive. The bioassay of the invention can be used to test effectiveness of individual compounds or mixtures of different compounds. In one embodiment, one or more flies are provided in a container that permits gas exchange. In one embodiment, the flies are Drosophila species, e.g., Drosophila melanogaster. In an exemplified embodiment, the flies are an insecticide-susceptible Canton-S strain of Drosophila. In another exemplified embodiment, the flies are a metabolically-resistant Hikone-R strain of Drosophila that exhibit elevated cytochrome P450 levels. In a further embodiment, the flies are a neurological mutant strain, for example, Rd1 or para-ts1 strains of Drosophila. A food substance is optionally provided in the container with the flies. The container with flies is then provided in a larger container that comprises a liquid absorbent material such as filter paper. The material is absorbed with some amount of a compound or a mixture of compounds to be screened for insecticidal activity. The compound(s) can be provided in solvent that exhibits little or no toxicity itself to the flies. Solvents contemplated within the scope of the invention include, but are not limited to, acetone, ethanol, methanol, methyl cellosolve, DMSO, and hexane. The compounds can also be provided in conjunction with a synergist compound, such as a compound that inhibits a cytochrome P450 enzyme (e.g., PBO) or that inhibits an esterase enzyme (e.g., DEF). Test compounds can be provided in solution at a concentration from about 10 μg/μl to about 1000 μg/μl. In an exemplified embodiment, the test compound is provided in solution at a concentration of about 100 μg/μl. The absorbent material can be treated with about 0.2 μl to about 200 μl of solution comprising the test compound(s). In an exemplified embodiment, the absorbent material is treated with about 2 μl to about 20 μl of test compound solution. The larger container is then sealed to contain the compound(s) within the container so that the flies are exposed to the compound(s). Flies are then exposed to the test compound(s) for a selected period of time, typically about 12 to 48 hours, and more typically about 24 hours. Mortality and/or knockdown of the flies exposed to test compound(s) is then determined.

Although Drosophila is not typically considered a pest species, it is highly amenable to large-scale insecticide screening operations; it is physiologically, biochemically and genetically similar to mosquitoes and flies of medical and agricultural importance; and it has well defined genetics that provides for testing upon strains with well defined backgrounds (ffrench-Constant et al., 2004). Furthermore, numerous insecticide-resistant Drosophila strains are available to the research community. For example, Drosophila strains are available that possess unique mutations that confer distinct types of physiological resistance, such as increased insecticide metabolism (ffrench-Constant et al., 2004; Pedra et al., 2004) and nervous system insensitivity to insecticides (ffrench-Constant et al., 1993; Martin et al., 2000).

Using a bioassay of the present invention, six compounds were identified that elicited highest levels of vapor toxicity (LC₅₀ range=400 to 1500 μg/jar). These compounds are menthofuran, benzothiophene, coumaran, butyl formate, hexyl formate and heptyl formate. Not included in this list is ethyl formate, a compound previously identified as being a highly effective fumigant for stored product applications; and which is registered for limited use in Australia (Caddick, 2004). Additionally, one volatile compound, ethylene glycol di-formate (EGDF), was also identified that rapidly caused 100% knockdown. However, EGDF treatment resulted in lower mortality after 24-hr than the other more effective test compounds noted above. Volatile compounds identified using the present invention can be formulated and utilized as aerosols, fumigants, or ultra low volume thermal fogs, or in slow release media such as fabric-treatment repellants, absorptive plastic devices, or ceramics for use in general pest control and public health applications.

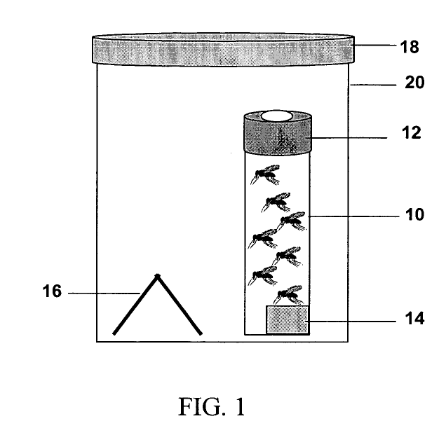

The subject invention also concerns an apparatus for conducting a bioassay for screening volatile compounds for activity against pests. One embodiment of the apparatus is shown in FIG. 1 and comprises a first container 10 for containing flies and that permits gas exchange. In one embodiment, the first container 10 is a container having at least one sealable open end and can be made of glass or other inert material wherein the open end can be covered with a material 12 (e.g., a fine mesh) that prevents flies from escaping but permits gas exchange. A food substance 14 that is a food source for the flies is optionally provided in the first container 10 with the flies. In use, the first container 10 with flies is provided in a releasably sealable second container 20 that can contain the first container 10 and that can also contain a liquid absorbent material 16 such as filter paper. In one embodiment, the second container 20 is an open-ended container made of glass or other inert material. In an exemplified embodiment, commercially available 0.5-L insect “killing jars” are used (Bio-Quip Products, Rancho Dominguez, Calif.) as the second container 20. The liquid absorbent material 16 can be absorbed with a suitable amount of a compound to be screened for insecticidal activity. The test compound can be provided in a solvent, such as acetone, ethanol, methanol, methyl cellosolve, DMSO, or hexane, or any other suitable solvent that exhibits little or no toxicity itself to the flies. The second container 20 comprising the first container 10 and the absorbent material 16 with the test compound applied thereon can be sealed, for example using a detachable lid 18, to contain the test compound within the containers so that the flies present in the first container 10 are exposed to molecules of the test compound present in the atmosphere of the containers.

The subject invention also concerns methods of using volatile compounds effective for killing pests. In one embodiment, a method of the invention comprises exposing or contacting a pest to an effective amount of a volatile compound of the invention. The compounds can be formulated in a composition and at a concentration effective for use as a pesticide or an insecticide. When a compound(s) of the present invention is to be used as an aerosol or a fumigant, the compound can be applied or used in an undiluted manner, or can be used and applied as a mix with an inert gas. The inert gas can be air, CO₂, N₂, or any other suitable gas. In one embodiment, a compound(s) of the invention is delivered via ultra low volume thermal fogging. In one embodiment, a compound(s) of the invention is applied in liquid form in an area or space in need of pest elimination and the active ingredients of the liquid allowed to vaporize. Apparatus for evaporative containment and release of volatile substances are known in the art (see, for example, U.S. Pat. No. 6,896,196). Compounds of the present invention can also be formulated for delivery via slow release media such as absorptive plastic devices, fabrics, and ceramics. Compounds of the present invention can be provided in combination with other pesticidal, insecticidal, and/or synergist compounds. In one embodiment, a synergist compound is one that inhibits a cytochrome P450 enzyme or an esterase enzyme. In an exemplified embodiment, the compound is PBO or DEF. In one embodiment, a volatile compound used in the methods of the present invention is a heterobicyclic compound. In specific embodiments, the compounds used in the methods are menthofuran, benzothiophene, coumaran, 9,9-difluoro-4-methyl-7-oxabicyclo[4.3.0]non-3-ene, and 4-methyl-7-oxabicyclo[4.3.0]non-1(6),3-diene. In another embodiment, a compound used in the methods is a formate ester. In specific embodiments, the compounds are methyl formate, ethyl formate, propyl formate, butyl formate, hexyl formate, heptyl formate, tert-butyl formate, ethylene glycol di-formate (EGDF), 1,2-propylene glycol diformate, 1,3-propylene glycol diformate, 1,4-propylene glycol diformate, and cyclopentyl formate. The methods of the present invention contemplate the use of any single compound or combination of compounds of the present invention. For example, in one embodiment, a method of the invention can use a combination of one or more heterobicyclic compounds and one or more formate ester compounds. Control of dipterans that are included within the scope of the invention include, but are not limited to, Aedes spp., Anopheles spp., Culex spp. (including Culex nigripalpus), Drosophila melanogaster, Musca spp. (including Musca domestica), Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio spp. (including Bibio hortulanus), Oscinella frit, Phorbia spp., Pegomyia hyoscyami, Ceratitus capitata, Dacus oleae, and Tipula paludosa.

The subject invention also concerns pesticidal formulations comprising volatile compounds, including heterobicyclic and aliphatic ester compounds. In one embodiment, the compounds are heterobicyclics. In specific embodiments, the compounds are menthofuran, benzothiophene, coumaran, 9,9-difluoro-4-methyl-7-oxabicyclo[4.3.0]non-3-ene, and 4-methyl-7-oxabicyclo[4.3.0]non-1(6),3-diene. In another embodiment, the compounds are formate esters. In specific embodiments, the compounds are methyl formate, ethyl formate, propyl formate, butyl formate, hexyl formate, heptyl formate, tert-butyl formate, ethylene glycol di-formate (EGDF), 1,2-propylene glycol diformate, 1,3-propylene glycol diformate, 1,4-propylene glycol diformate, and cyclopentyl formate. Formulations of the present invention contemplate the use of any single compound or combination of compounds of the present invention. For example, in one embodiment, formulations of the invention can comprise a combination of one or more heterobicyclic compounds and one or more formate ester compounds. Compounds of the present invention can also be formulated for delivery via slow release media such as absorptive plastic devices, fabrics, and ceramics. In one embodiment, a pesticidal formulation is formulated as an aerosol or a fumigant. The formulation can optionally comprise an inert gas, including, for example, air, CO₂, N₂, or any other suitable gas. In another embodiment, the formulation is in liquid form. In a further embodiment, a pesticidal formulation of the invention can comprise a synergist compound. In one embodiment, the synergist compound is one that inhibits a cytochrome P450 enzyme or an esterase enzyme. In an exemplified embodiment, the synergist is PBO or DEF.

An insecticidal compound's propensity to volatilize plays at least a minor role in vapor phase toxicity (Brown et al., 1951). However, as data presented herein shows, other structural factors also contribute to the widely varying toxicity of low molecular weight insecticides from both the heterobicyclic and ester classes. With respect to the heterobicyclic compounds, two structure-activity relationship trends are apparent. First, when no peripheral methyl groups are present, sulfur in the first position of the furan ring is associated with greater toxicity than if oxygen or nitrogen are in this position (i.e., benzothiophene>coumaran>indole). Second, when oxygen is in the first position of the furan ring and peripheral methyl branches are present, opposing methyl branches are associated with greater toxicity than adjacent methyl branches (i.e., menthofuran>coumaran). Because a mix of menthofuran stereo-isomers was evaluated, it is not possible to comment on the role of chirality in heterobicyclic toxicity.

With respect to the aliphatic ester compounds, several structure-activity relationships are also apparent. First, as aliphatic chain length on the acid group increases, toxicity generally decreases. Clearly, the formates elicited the highest toxicity of all compounds tested from the ester group (i.e., formates>acetates>propionates>butyrates>valerates). Second, within the formate group, aliphatic chain lengths with 4-7 carbons had highest toxicity, with butyl formate being the most toxic. Finally, although EGDF elicited only knockdown activity, it was highly effective at doing so. Interestingly, butyl formate would apparently be released upon hydrolysis of a single EGDF ester linkage. By additional hydrolysis, the butyl formate could be converted to formic acid, which is presumably the toxic metabolite liberated from all the formates (Nicholls, 1975). Thus, one molecule of EGDF could conceivably liberate two formic acid molecules.

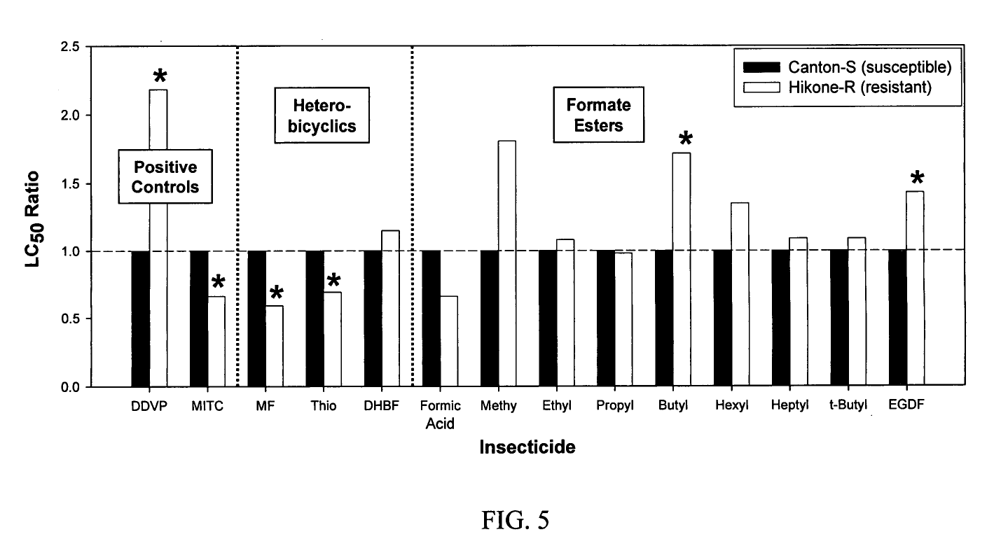

With respect to the heterobicyclics, two of these compounds (menthofuran and benzothiophene) were the most toxic materials evaluated in our study. Both of these compounds, along with the less effective compound coumaran, share a basic structural feature in common that consists of adjacent five- and six-member rings. Of these three compounds, only menthofuran has been previously evaluated for its toxicity to insects. Gunderson et al. (1986) determined that menthofuran was toxic to two lepidopteran insects, Spodoptera eridanea and S. frugiperda, with S. eridanea being the most susceptible. Upon further examination, it was determined that the greater susceptibility in S. eridanea correlated with higher constitutive cytochrome P450 activity, and that this activity was highly inducible by menthofuran exposure (Gunderson et al., 1986). This finding suggests that menthofuran is activated to a more potent form by P450-based oxidation, and that insects resistant to other insecticides by P450 oxidation may be more susceptible to menthofuran. Indeed, in ongoing studies it has been observed that a Drosophila strain with elevated P450 is significantly more susceptible to menthofuran than the Canton-S strain used in the present study (FIG. 5).

The relationship of fumigant toxicity to volatility factors such as molecular weight, boiling point and diffusion rate are considered only partially responsible for acute toxicity (Brown, 1951; Tattersfield et al., 1920). Results of regression analyses herein concur with the idea that physical properties which affect volatility only weakly correlate with insecticidal activity. In this respect, structure-activity comparisons suggest several additionally important structural features for consideration when designing novel volatile insecticides. Thus, volatility is important, but so are other structural features that influence active site interactions, toxin activation and detoxification, to name a few.

To summarize: (i) active compounds identified include heterobicyclics (e.g., menthofuran, benzothiophene and coumaran) and formate esters (e.g., butyl-, hexyl- and heptyl-formate), (ii) bioassays with the enzymatically-resistant Hikone-R strain allowed us to identify a role for cytochrome P450-based metabolism in detoxification of formate esters and in the activation of heterobicyclic compounds, (iii) bioassays using the P450 inhibitor PBO allowed us to identify P450-based detoxification of heterobicyclics, as well as P450-based activation of some formate esters, (iv) bioassays using the esterase inhibitor DEF allowed us to identify esterase-based activation of some formate esters, (v) bioassays with neurological mutants allowed us to determine that insecticide-resistance-conferring point mutations in insect sodium and chloride channels confer enhanced susceptibility to heterobicyclic insecticides, and (vi) finally, neurological mutant bioassays allowed us to determine that formate ester insecticides (and their toxic metabolite formic acid) are active at the Drosophila chloride channel.

### Materials and Methods for Examples 1 to 3

Fly Straining and Rearing.

The insecticide-susceptible Canton-S strain of Drosophila was obtained from the Bloomington Drosophila stock center (Indiana University, Bloomington, Ind.), and used exclusively in all studies. Flies were reared in 100-ml vials capped with acetate plugs (Fisher Scientific; Suwannee, Ga.) on a JAZZ-MIX diet (Fisher) prepared with a 2:1 ratio of water to apple juice. Flies were reared on a 12:12 photocycle at 24° C. and ambient relative humidity. Mixed-sex adults, less than 1-wk old, were used in bioassays.

Bioassays

Flies were briefly anesthetized in rearing vials with a pulse of CO₂ and transferred to a CO₂ flowbed (Genesee Scientific; San Diego, Calif.). Using a camel hair brush and a 5×5 cm sheet of rice paper, ten flies were placed into 7.0 ml dram vials. Prior to adding flies, each vial received a 0.5 cm³ block of rearing diet that had been sufficiently dried on a paper towel to remove excessive moisture. The vials were then capped with open-top septum caps (Fisher) that were covered with fine mesh. The mesh was applied to the septum caps in advance using hot glue; it prevented fly escape but readily permitted gas exchange. The flies were allowed 1-hr to recover from the CO₂ anesthesia. After one hour, single vials with flies were placed into 0.5 L glass jars, along with a filter paper tent (see FIG. 1). In a fume hood, filter paper tents were treated with insecticide dilutions or acetone for controls. The jars were then rapidly closed tightly with a metal lid. After 24 hours of exposure, mortality was scored with the aid of a magnifying glass. Flies were considered dead only when they showed a complete lack of movement.

All test compounds and solvents were >99% purity, and were purchased from Sigma-Aldrich Chemical (Milwaukee, Wis.). See Table 1 for a listing of the test compounds and their structures. Insecticide stock solutions were prepared at a standard concentration of 100 μg/μl in analytical grade acetone. For liquid insecticidal compounds, density in mg/μl was used to calculate weight on a per-volume basis. Stock solutions were held at −20° C. in sealed amber vials. In bioassay jars, filter paper tents were treated with stock volumes ranging from 2 to 20 μl, depending on the inherent toxicity of the test compound. These volumes of insecticide stock provided test concentrations of 200 to 2000 μg insecticide per replicate jar. A range of 4-5 concentrations plus a control were tested for each insecticide, and each range was repeated five times over at least three days. Controls received a volume of acetone identical to the highest insecticide volume that was tested (i.e., 10 μl for menthofuran; 20 μl for all other insecticides). Between uses, bioassay jars and lids were washed in a dishwasher, then baked 12-16 hr at 90° C. in a drying oven.

Data Analysis

All data analysis was performed using SAS statistical software (SAS Institute, SAS systems for linear models, Cary, 2000) as demonstrated in previous reports (Scharf et al., 1995; Scharf et al., 1999). Probit and regression analyses were performed using the PROC PROBIT and PROC REG procedures, respectively. Abbott's transformation was automatically performed as part of the PROBIT procedure to correct for control mortality in the few instances when it was encountered. If control mortality ever exceeded 10% in a given replicate, that replicate was discarded.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLE 1

### Bioassay Development and Optimization

Several bioassay configurations were compared for exposing and holding test insects. The optimal bioassay configuration is shown in FIG. 1. This configuration, which permits 100% fly survival for >72-hr under control conditions, involves placing flies in 7-ml dram vials with a 0.5 cm³ block of diet, capping the vials with vented caps, and then sealing the vials in 0.5 L glass jars with metal lids.

Using the optimized bioassay conditions, seven solvent carriers were tested for their relative toxicity to test insects. The solvent carriers that were tested included acetone, ethanol, methanol, methyl cellosolve, DMSO, hexane and isopropanol. Only isopropanol elicited mortality, which was severe (i.e., 100% mortality). No mortality was observed for the remaining solvents, as well as untreated controls. Because of its broad use as a solvent carrier in insecticide efficacy research, acetone was chosen as the standard solvent for use in the volatility bioassay. Additional investigations determined that only acetone volumes above 22 μl caused significant mortality in test insects (results not shown).

### EXAMPLE 2

### Evaluation of Candidate Insecticidal Compounds

Thirty volatile low molecular weight compounds with suspected insecticidal activity were identified and purchased from commercial sources (see Table 1 for structures). The majority of these compounds are liquids (28 of 30); only benzothiophene and indole are solids. These materials were either dissolved or diluted in acetone at a standard concentration of 100 μg/μl and applied to bioassay jars in volumes under 20 μl.

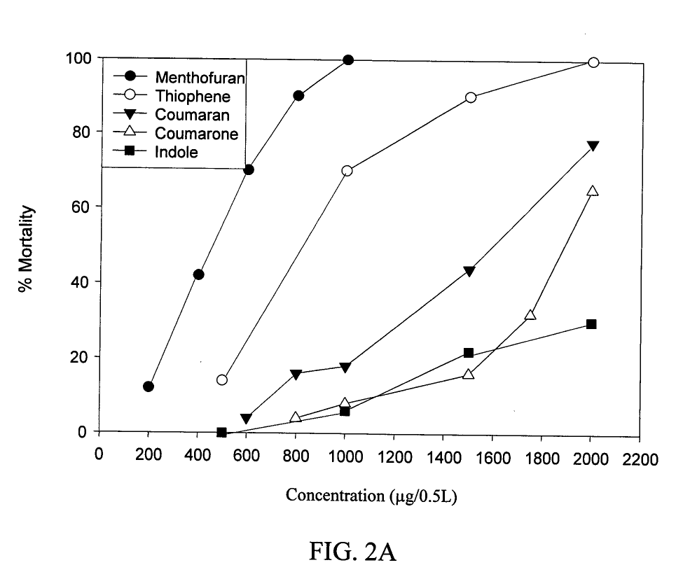

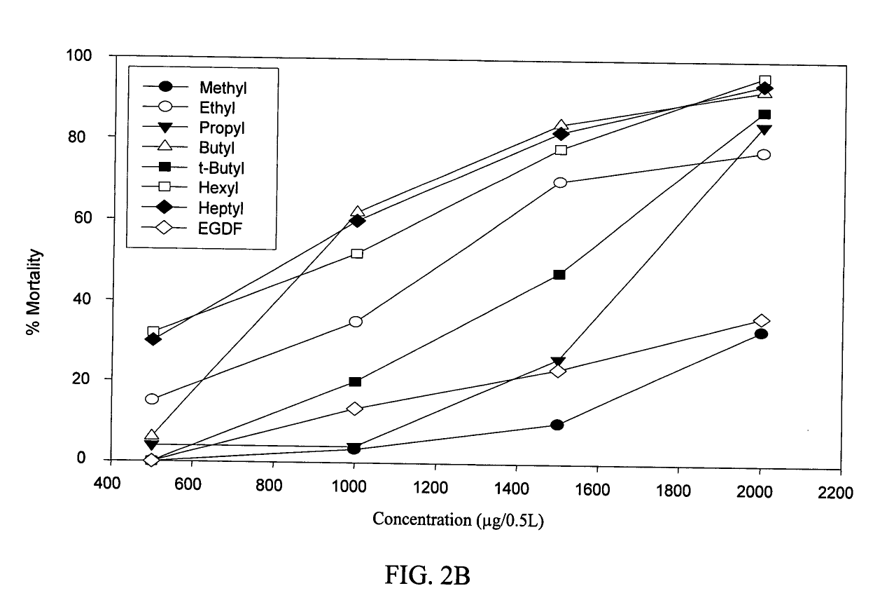

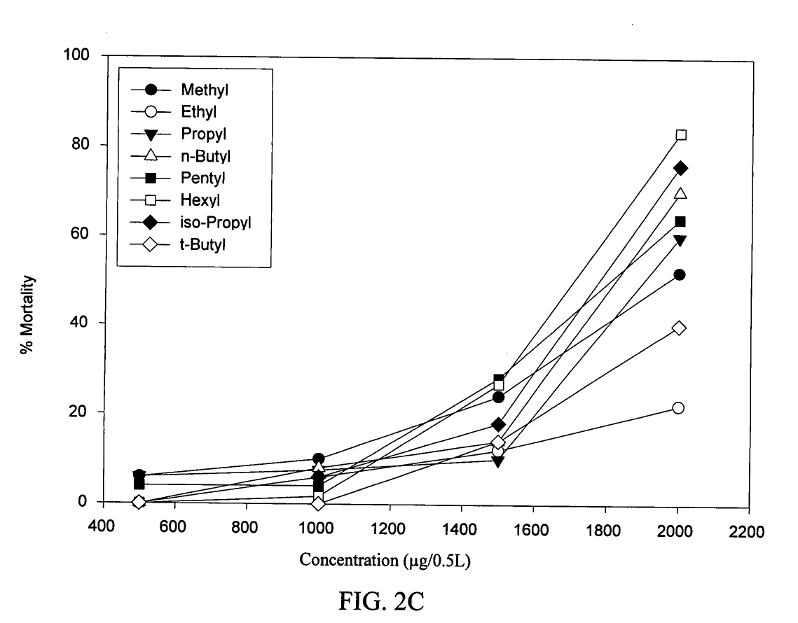

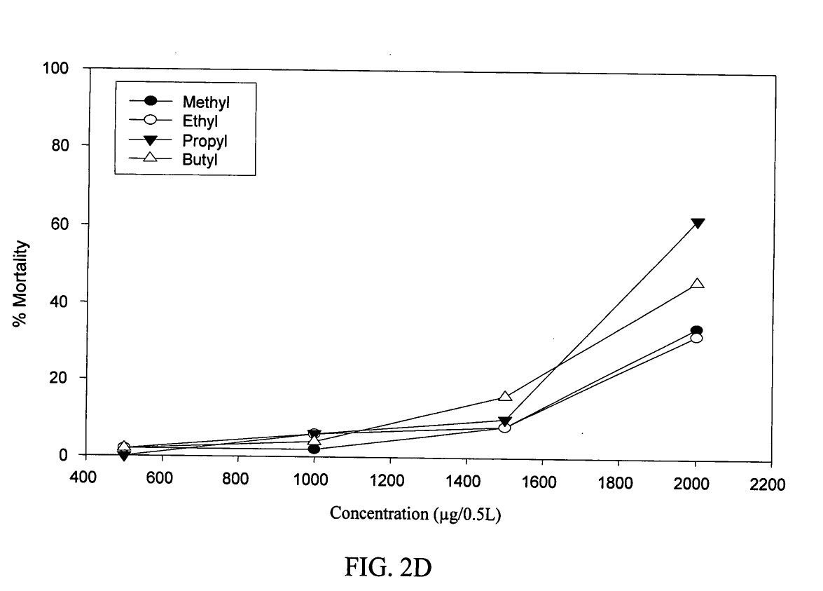

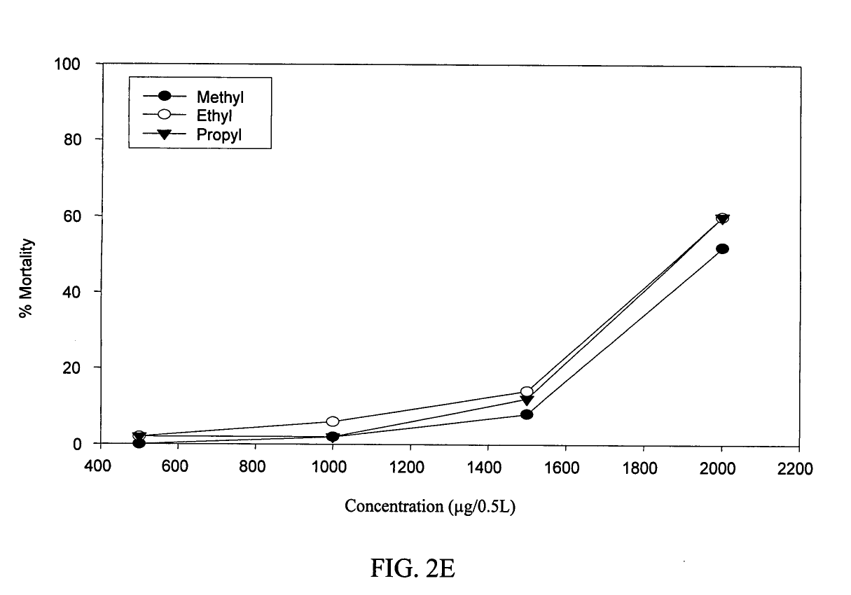

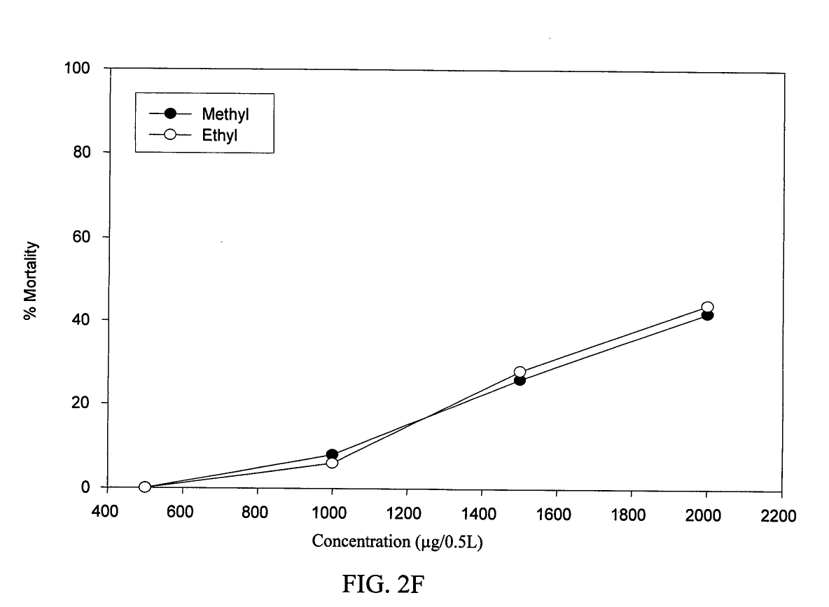

Concentrations of the thirty compounds ranging between 200 and 2000 μg/jar were tested, which equated with between 2 and 20 μl of stock solution being applied per jar. These concentrations provided a linear concentration-mortality relationship for all insecticides tested (FIG. 2). All data points shown in FIG. 2 were subjected to probit analysis.

Probit analysis results are shown in Table 1. The data that are reported include sample size (n), slope, goodness-of-fit characteristics (chi-square), and LC₅₀ and LC₉₀ estimates with 95% confidence limits. In general, as shown by chi-square results, mortality followed an expected dose-mortality relationship most of the time. In some instances where chi-square values were moderately high (i.e., >5.0), there were minor impacts on LC confidence limits (i.e., note chi-square and confidence limits for coumaran and butyl formate). However, in other cases poor model fit resulted in both excessive chi-square values and an inability to calculate confidence limits around LC estimates (i.e., note chi-square and confidence limits for propyl formate, propyl propionate, ethyl butyrate and propyl butyrate). It is striking that propyl esters were involved in 3 of 4 cases of excessive poor model fit. This supports the idea that poor insecticidal activity causes poor model fit, rather than other uncontrollable bioassay conditions.

Overall, the best performing volatile insecticides were the heterobicyclics menthofuran and benzothiophene (LC₅₀=414.8 and 802.1 μg/jar, respectively). These two compounds were followed by the esters butyl-, hexyl- and heptyl formate (LC₅₀=913.1, 1140.0 and 1357.0 μg/jar, respectively), and then the heterobicyclic coumaran (LC₅₀=1479.0 μg/jar). All other tested compounds had LC₅₀ estimates ranging from 1500 to above 3500 μg/jar. Finally, although the ester compound ethylene glycol diformate (EGDF) had a poor LC₅₀ of 2500 μg/jar, it elicited 100% knockdown by 2-hr that lasted through 24-hr in all bioassay replicates.

### EXAMPLE 3

### Evaluation of the Role of Volatility in Toxicity

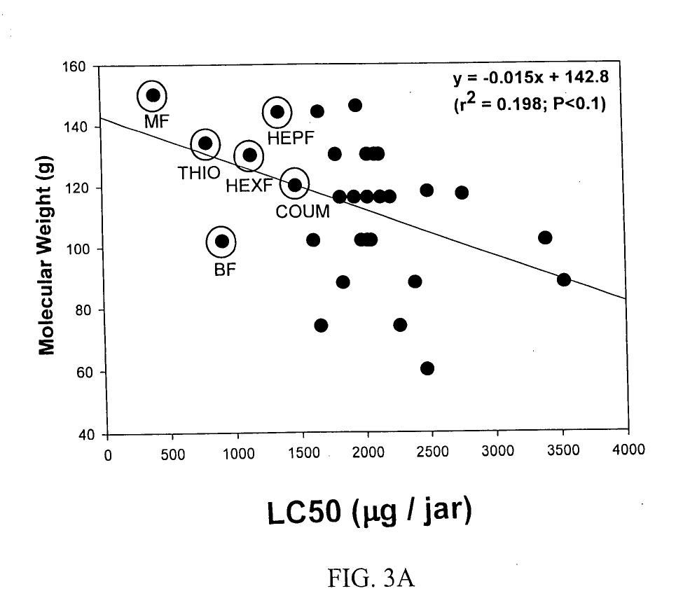

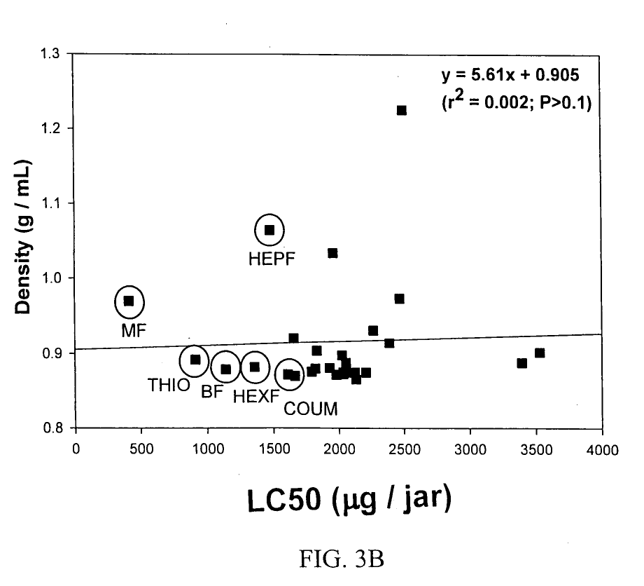

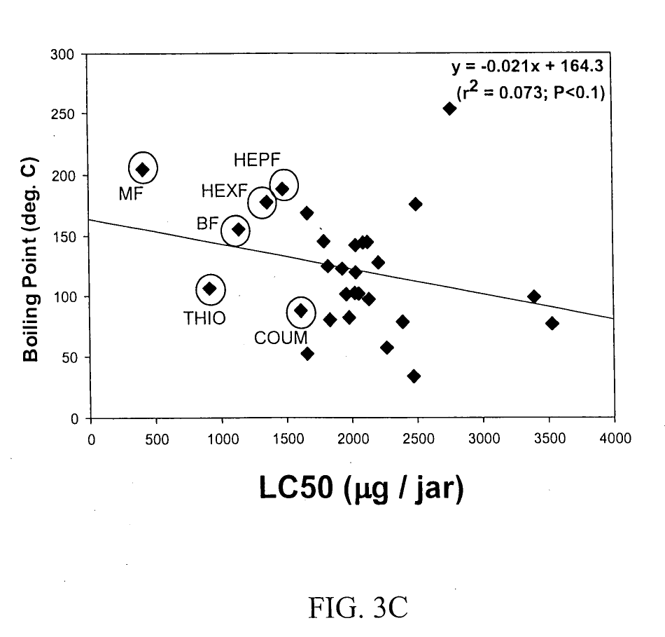

Linear regression analyses were performed that compared LC₅₀ versus molecular weight, density and boiling point of the 30 insecticidal compounds (FIG. 3). These properties were chosen for analysis because they are predictors of volatility, and because vapor pressures are not available for most of the compounds. All three regressions were weak (r²<0.2). In spite of this, the two regressions of LC₅₀ versus molecular weight and LC₅₀ versus boiling point were significant, but only at the α=0.10 level. Also, as can be seen from the regression plots, the most effective insecticidal compounds tended to cluster together in the portion of the curve representing the greatest volatility.

TABLE 1
Structures and toxicity of volatile insecticides to insecticide-susceptible Drosophila melanogaster, as
determined by probit analysis. The LC₅₀s of the most effective compounds are highlighted.

| | | Slope ± | Chi- | LC₅₀ (95% CL)^{c} | LC₉₀ (95% CL)^{c} |
|---|---|---|---|---|---|
| Compound | N | Std. Error^{a} | Square^{b} | [μg/jar] | [μg/jar] |
| Heterobicyclics | | | | | |
| MENTHOFURAN | 250 | 4.42 ± 0.47 | 5.00 | 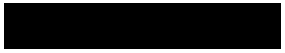 | 808.7 (711.5 − 926.0) |
| 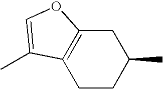 | | | | | |
| BENZOTHIOPHENE | 200 | 5.37 ± 0.61 | 1.43 | 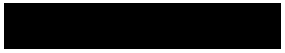 | 1390.0 (1234.0 − 1634.0) |
| 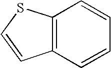 | | | | | |
| COUMARAN | 250 | 4.50 ± 0.55 | 3.36 | 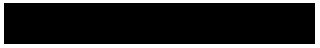 | 2848.0 (2366.0 − 3804.0) |
| 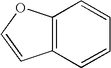 | | | | | |
| DIMETHYL-COUMARONE | 250 | 5.25 ± 1.45 | >5.00* | 1960.0 (1543.0 − 2452.0) | 3434.0 (2278.0 − >10,000) |
| 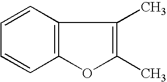 | | | | | |
| INDOLE | 150 | 3.31 ± 1.08 | 0.49 | 2769.0 (2137.0 − 7873.0) | 6739.0 (3759.0 − >90,000) |
| 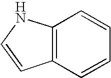 | | | | | |
| Low Molecular Weight | | | | | |
| Esters: Formates | | | | | |
| METHYL | 120 | 3.27 ± 0.95 | 1.09 | 2471.0 (1915.0 ± 5336.0) | 6094.0 (3532.0 − >40,000) |
| 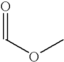 | | | | | |
| ETHYL | 120 | 5.18 ± 1.07 | 1.03 | 1656.0 (1486.0 − 1917.0) | 2926.0 (2365.0 − 4705.0) |
| 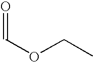 | | | | | |
| PROPYL | 150 | 23.24 ± 3.22 | 1.92 | 1833.0 (1787.0 − 1884.0) | 2081.0 (2005.0 − 2208.0) |
| 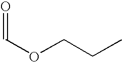 | | | | | |
| BUTYL | 230 | 5.43 ± 0.62 | 1.97 | 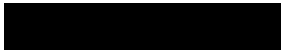 | 1572.0 (1419.0 − 1811.0) |
| 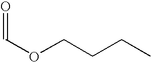 | | | | | |
| HEXYL | 200 | 9.23 ± 1.34 | 1.56 | 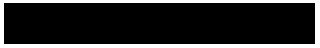 | 1570.0 (1462.0 − 1761.0) |
| 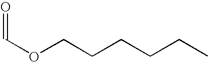 | | | | | |
| HEPTYL | 250 | 8.54 ± 1.21 | 1.33 | 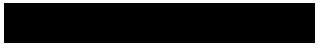 | 1917.0 (1793.0 − 2133.0) |
| 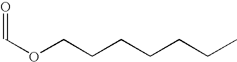 | | | | | |
| Low Molecular Weight | | | | | |
| Esters: Formates | | | | | |
| t-BUTYL | 120 | 22.21 ± 3.57 | 2.72 | 1981.0 (1917.0 − 2048.4) | 2262.0 (2166.0 − 2435.0) |
| ETHYLENE GLYCOL DI- | 120 | 5.04 ± 1.66 | 0.49 | 2500.0 (2037.0 − 5546.0) | 4492.0 (2980.0 − >25,000) |
| 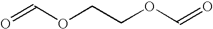 | | | | | |
| Low Molecular Weight | | | | | |
| Esters: Acetates | | | | | |
| METHYL | 200 | 2.86 ± 0.91 | >5.00* | 2268.0 (ND) | 6346.0 (ND) |
| 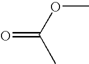 | | | | | |
| ETHYL | 200 | 3.08 ± 0.94 | 0.48 | 3530.0 (2488.0 − 12723.0) | 9204.0 (4619.0 − >10,000) |
| 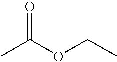 | | | | | |
| PROPYL | 330 | 5.08 ± 3.86 | >5.00* | 2056.0 (ND) | 3673.0 (ND) |
| 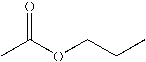 | | | | | |
| n-BUTYL | 100 | 12.84 ± 2.31 | >5.00* | 1821.0 (1730.0 − 1930.0) | 2291.0 (2115.0 − 2668.0) |
| 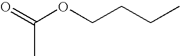 | | | | | |
| PENTYL | 150 | 7.09 ± 1.20 | 0.07 | 1792.0 (1658.0 − 1987.0) | 2716.0(2347.0 − 3579.0) |
| 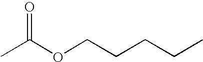 | | | | | |
| HEXYL | 180 | 11.11 ± 1.52 | 1.47 | 1666.0 (1582.0 − 1755.0) | 2172.0 (2019.0 − 2437.0) |
| 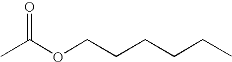 | | | | | |
| ISO-PROPYL | 150 | 7.52 ± 1.13 | 0.01 | 1611.0 (1458.0 − 1782.0) | 2385.0 (2104.0 − 2917.0) |
| 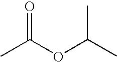 | | | | | |
| t-BUTYL | 150 | 7.69 ± 1.78 | 0.53 | 2134.0 (1943.0 − 2598.0) | 3131.0 (2580.0 − 5145.0) |
| 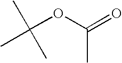 | | | | | |
| Low Molecular Weight | | | | | |
| Esters: Propionates | | | | | |
| METHYL | 150 | 6.05 ± 1.50 | 0.94 | 2391.0 (2076.0 − 3433.0) | 3893.0 (2925.0 − 8918.0) |
| 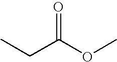 | | | | | |
| ETHYL | 200 | 2.87 ± 0.77 | 4.05 | 3395.0 (2439.0 − 9039.0) | 9483.0 (4885.0 − >70,000) |
| 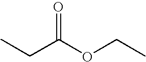 | | | | | |
| PROPYL | 200 | 6.95 ± 2.55 | >5.00* | 1931.0 (ND) | 2952.0 (ND) |
| 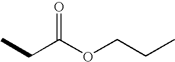 | | | | | |
| Low Molecular Weight | | | | | |
| Esters: Propionates | | | | | |
| BUTYL | 150 | 5.74 ± 1.24 | 6.63 | 2126.0 (1894.0 − 2670.0) | 3555.0 (2785.0 − 6340.0) |
| 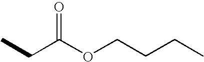 | | | | | |
| Low Molecular Weight | | | | | |
| Esters: Butyrates | | | | | |
| METHYL | 200 | 8.40 ± 1.67 | 3.13 | 2026.0 (1872.0 − 2314.0) | 2878.0 (2466.0 − 4027.0) |
| 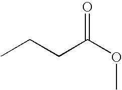 | | | | | |
| ETHYL | 200 | 4.72 ± 2.36 | >5.00* | 2034.0 (ND) | 3799.0 (ND) |
| 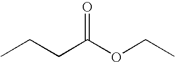 | | | | | |
| PROPYL | 200 | 5.44 ± 4.90 | >5.00* | 2035.0 (ND) | 3498.0 (ND) |
| 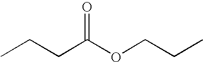 | | | | | |
| Low Molecular Weight | | | | | |
| Esters: Valerates | | | | | |
| METHYL | 200 | 4.25 ± 0.91 | 0.22 | 2209.0 (1897.0 − 2997.0) | 4422.0 (3184.0 − 9603.0) |
| 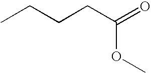 | | | | | |
| ETHYL | 200 | 4.70 ± 1.00 | 0.49 | 2092.0 (1837.0 − 2670.0) | 3915.0 (2956.0 − 7415.0) |
| 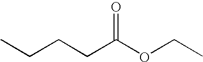 | | | | | |

^{a}Slope of the best − fit probit mortality line
^{b}Pearson's Chi-square goodness-of-fit test., testing whether the data fit an expected concentration-mortality probit model. Values followed by “*” indicate a lack of fit relative to an expected concentration-mortality probit model.
^{c}LC values and 95% confidence limits (CL) are expressed in μg insecticide per 0.5 liter of headspace. “ND” indicates that confidence limits were not determinable due to lack of fit by raw data to probit model (i.e., Chi-square >5.0).

### Materials and Methods for Examples 4 to 10

Drosophila Strains and Rearing

Four Drosophila strains were used, all obtained from the Bloomington Drosophila Stock Center (Indiana University; Bloomington, Ind.). The Canton-S strain was used as the insecticide-susceptible standard. The Hikone-R strain is metabolically-resistant with elevated cytochrome P450 levels (Waters et al., 1984; Sundseth et al., 1989; Le Goff et al., 2003; Festucci-Buselli et al., 2005). Hikone-R is resistant to a number of insecticides, including malathion, DDT and neonicotinoids (Sundseth et al., 1989; Daborn et al., 2001). Two resistant neurological-mutant strains were also tested. The first of these is “Rd1” (ffrench-Constant et al., 1990; ffrench-Constant and Roush, 1991), which possesses a GABA-gated chloride channel point mutation (ffrench-Constant et al., 1993) that confers cross-resistance to cyclodiene and phenylpyrazole insecticides (Bloomquist, 2000). The second neurological strain is “para-ts1” (Suzuki, 1971), which possesses a sodium channel point mutation that causes temperature sensitivity (Loughney et al., 1989), knock-down resistance to DDT (Pittendrigh et al., 1997), and hyper-susceptibility to pyrethroids such as deltamethrin (Pedra et al., 2004). Flies were reared in 100-ml vials capped with acetate plugs (Fisher Scientific; Suwanee, Ga.) on a commercial diet (JAZZ-MIX; Fisher Scientific). Flies were reared on a 12:12 photocycle at 24° C. and 60% relative humidity. Mixed-sex adults, less than 1-wk old, were used in bioassays.

Chemicals

All experimental materials were purchased from Sigma-Aldrich-Fluka (Milwaukee, Wis.) and were of 99% purity or greater. DDVP and MITC were purchased from ChemService (West Chester, Pa.) and were >98% purity. All volatile insecticide stocks were prepared at 100 μg/μl in analytical grade acetone. Rather than weigh the highly volatile liquid insecticides (i.e., all compounds except benzothiophene), weight was determined based on the density-volume relationship of each compound. Four serial dilutions were prepared and tested for each insecticide as described previously (Scharf et al., 2006). The insecticide synergists PBO (piperonyl butoxide) and DEF (SSS-tributyl-phosphoro-trithioate) were obtained from Fluka Chemical Co. (Basel, Switzerland) and Mobay Chemical Co. (Kansas City, Mo.). Both synergists were >95% purity. Synergist stocks were prepared at 100 μg/ml in analytical grade acetone.

Bioassays

Volatility bioassays were conducted exactly as described in a previous report (Scharf et al., 2006). Briefly, bioassays took place in 0.5-l glass jars with metal lids. Mixed-sex flies were isolated from lab colonies and placed in 4-ml dram vials in groups of ten, along with a dried piece of laboratory diet. The dram vials were capped with open-top septum caps that were covered with fine mesh (held in place by non-toxic glue). For bioassays, the assembled dram vials were placed into the 0.5-l jars along with a folded filter paper “tent” (Whatman #1; Vineland, N.J.). The filter paper was treated with either an insecticide dilution or acetone, the jar was sealed with the metal lid, and the bioassay proceeded for 24-hr at room temperature. Mortality was scored based on a complete lack of movement by the flies. Four concentrations plus an acetone control were tested for all insecticides. Between five and ten replicates were performed for each concentration range on each strain-insecticide combination. Synergist bioassays were performed with a slight modification. Synergist stocks were applied to dram vials at 100 μl per vial to provide assay concentrations of 10 μg per vial. Preliminary investigations showed that this concentration causes no mortality under bioassay conditions after 24-hr of exposure. After treatment, vials were held at an angle in a fume hood and rotated ¼-turn each minute until the acetone evaporated. Flies and diet were added as above and held for 1-hr. Assays were initiated, run, and scored as above.

Data Analysis

Probit analysis was performed using PROC PROBIT in the SAS software package (SAS Institute; Cary, N.C.). If control mortality ever exceeded 10% in a given replicate, that replicate was discarded. Toxicity and synergist ratios at LC₅₀ were compared statistically using the calculation described by Robertson and Preisler (1992). Using this procedure, ratios with 95% confidence intervals were calculated using a spreadsheet-based program. With this calculation, if confidence intervals include 1.0 then ratios are considered non-significant (p>0.05; Robertson and Preisler, 1992). Ratio confidence intervals that do not include 1.0 are considered significant (p<0.05).

### EXAMPLE 4

### Baseline Toxicity in an Insecticide-Susceptible Strain

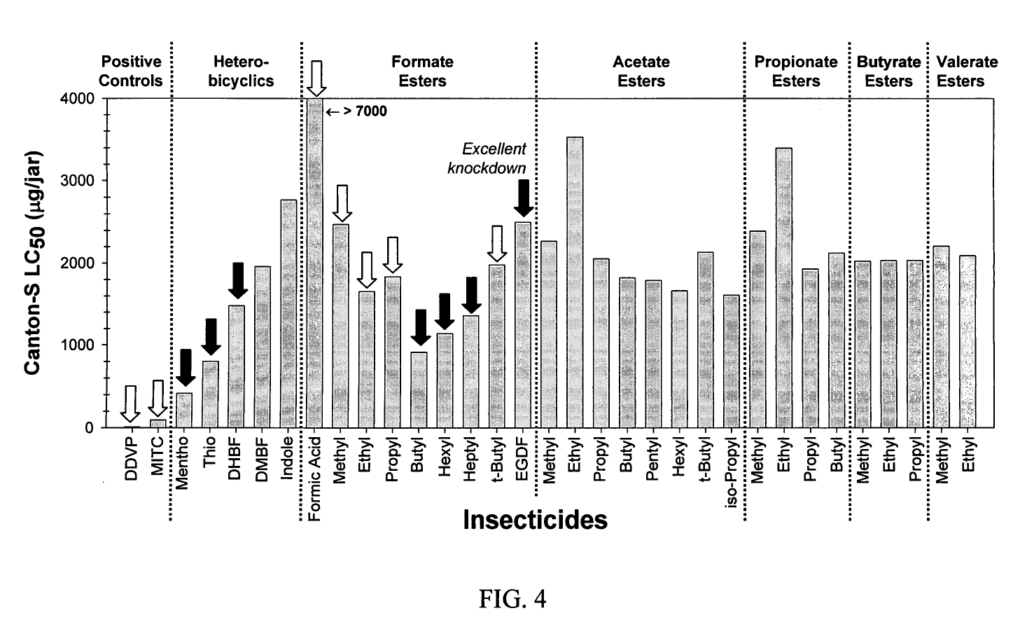

Volatility bioassays were initially used to evaluate 30 candidate insecticidal compounds against the insecticide-susceptible Canton-S strain (FIG. 4). The two established fumigant insecticides DDVP and MITC were also tested, as well as formic acid, which is a possible active metabolite of the formate esters. See Table 2 for detailed probit analysis results and Scharf et al., (2006) for plots of raw bioassay data. The thirty compounds displayed varying degrees of toxicity. The most effective insecticides were from the heterobicyclic group (mentho- and benzothiophene), followed by three formate esters (butyl-, hexyl- and heptyl formate), then the heterobicyclic dihydrobenzofuran (DHBF) and the formate ester ethylene glycol diformate (EGDF). Although EGDF did not cause high acute mortality, it did elicit 100% knockdown. DDVP and MITC exhibited extremely high toxicity in comparison to all other test compounds. Other aliphatic esters from the acetate, propionate and butyrate groups were not as effective as the formate esters.

### EXAMPLE 5

### Bioassays with an Enzymatically-Resistant Strain

Because of the broad role of enzyme-based detoxification in insecticide resistance, particularly cytochrome P450, we compared insecticide toxicity in the metabolically resistant Hikone-R strain to Canton-S (FIG. 5). Detailed Hikone-R probit analysis results can be found in Table 2. Hikone-R shows significant resistance to DDVP, butyl formate and EGDF, and non-significant tolerance to DHBF, hexyl-, heptyl- and t-butyl-formate. Interestingly, relative to Canton-S, Hikone-R has significantly enhanced susceptibility to MITC, mentho- and benzothiophene, as well as non-significant tolerance to formic acid. These findings imply a role for cytochrome P450 in detoxification of DDVP, butyl formate and EGDF, and also suggest potential oxidation-based cross-resistance between DDVP and formate esters. Enhanced susceptibility results imply that MITC, mentho- and benzothiophene are activated to more toxic metabolites by cytochrome P450.

With respect to structure in general, the heterobicyclics are characterized by adjacent 5- and 6-member ring structures, while the formate esters consist of formic acid connected via an ester linkage to alkyl chains of 1-7 units. EGDF is distinct from the other esters in that it contains two formic acid groups connected via ester linkages to a central ethyl chain.

### EXAMPLE 6

### Synergist Bioassays

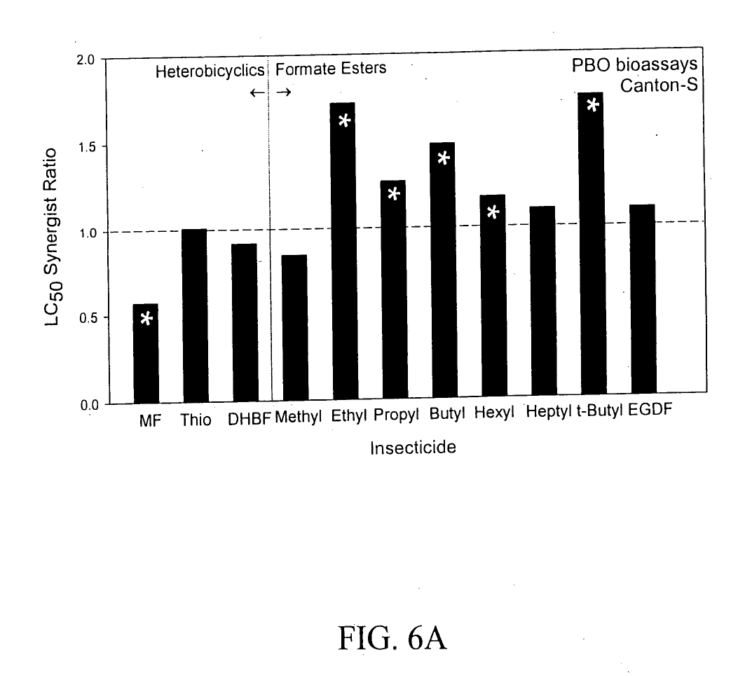

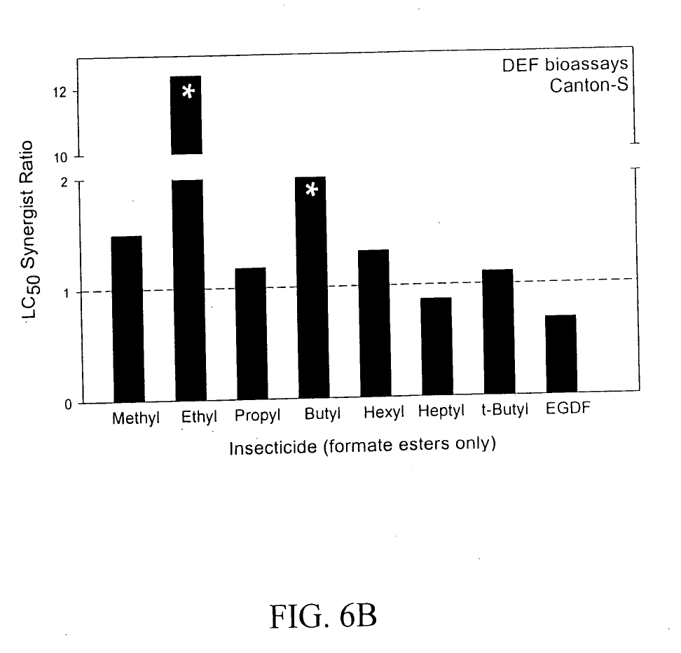

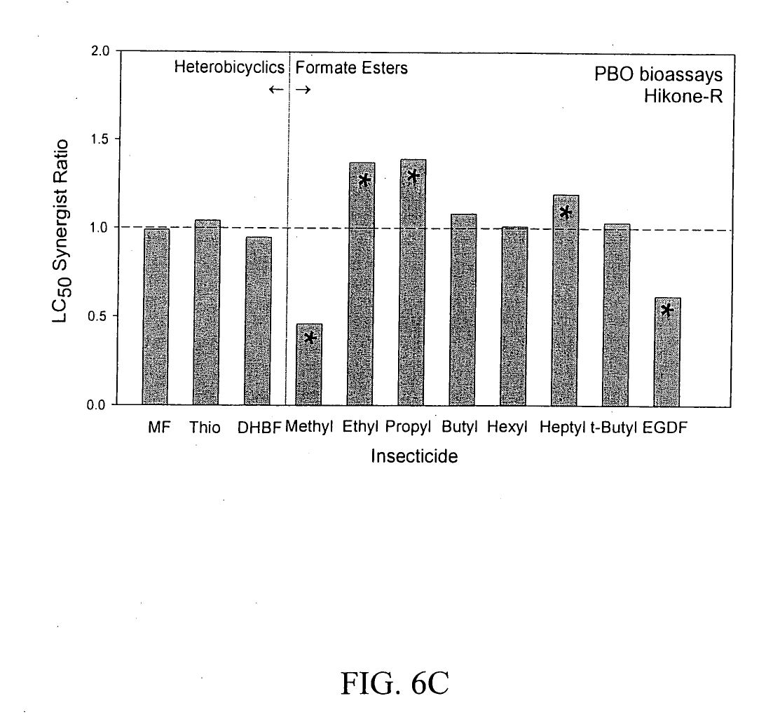

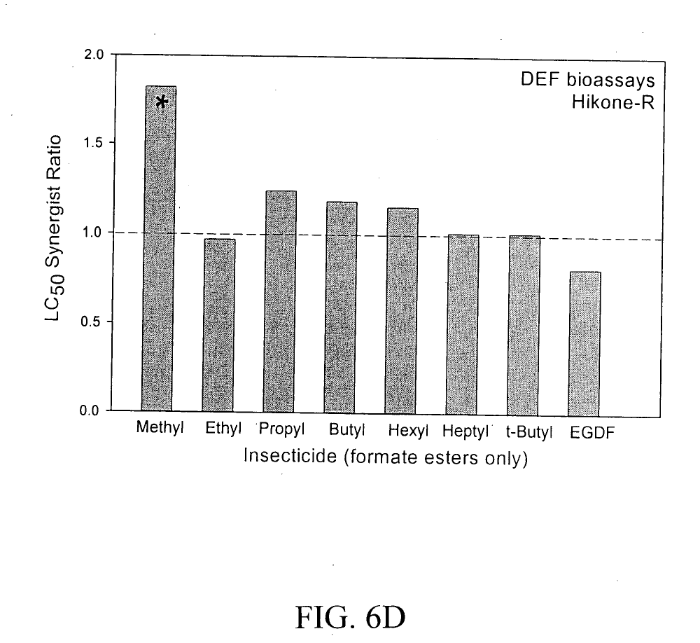

To further examine potential impacts on toxicity by metabolic mechanisms, we tested the synergists PBO and DEF, which act by inhibiting cytochrome P450 and esterase enzymes (respectively). Thus, PBO and DEF can reveal the contributions of P450s and esterases to xenobiotic detoxification or activation. Probit analysis summaries from synergism studies are provided in Table 3. These results indicate differing results between Canton-S and Hikone-R that are explained by the differing detoxification capabilities between these two strains. Depending on the insecticide, these results showed varying degrees of synergism (=increased toxicity; detoxification) or antagonism (=reduced toxicity; activation) (FIG. 6). In general, irrespective of fly strain PBO results imply that P450 plays a significant role in detoxifying menthofuran, methyl formate and EGDF, while P450 contributes significantly to the metabolic activation of ethyl, propyl, butyl, hexyl, heptyl and t-butyl formate (FIGS. 6A & 6B). Alternatively, findings for DEF imply that esterases play no significant role in formate detoxification. Also, while esterases appear to contribute only weakly to activation of nearly all formate esters, they only play significant roles in the activation of methyl, ethyl and butyl formate (FIGS. 6C & 6D).

### EXAMPLE 7

### Bioassays with Insecticide-Resistant Neurological Mutant Strains

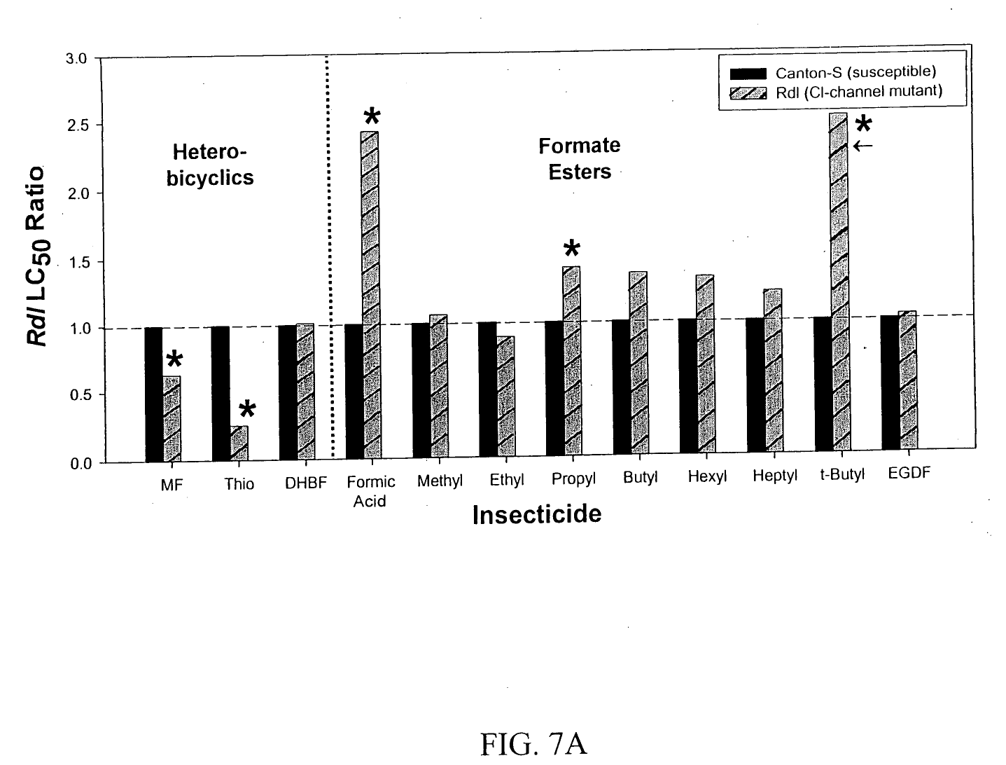

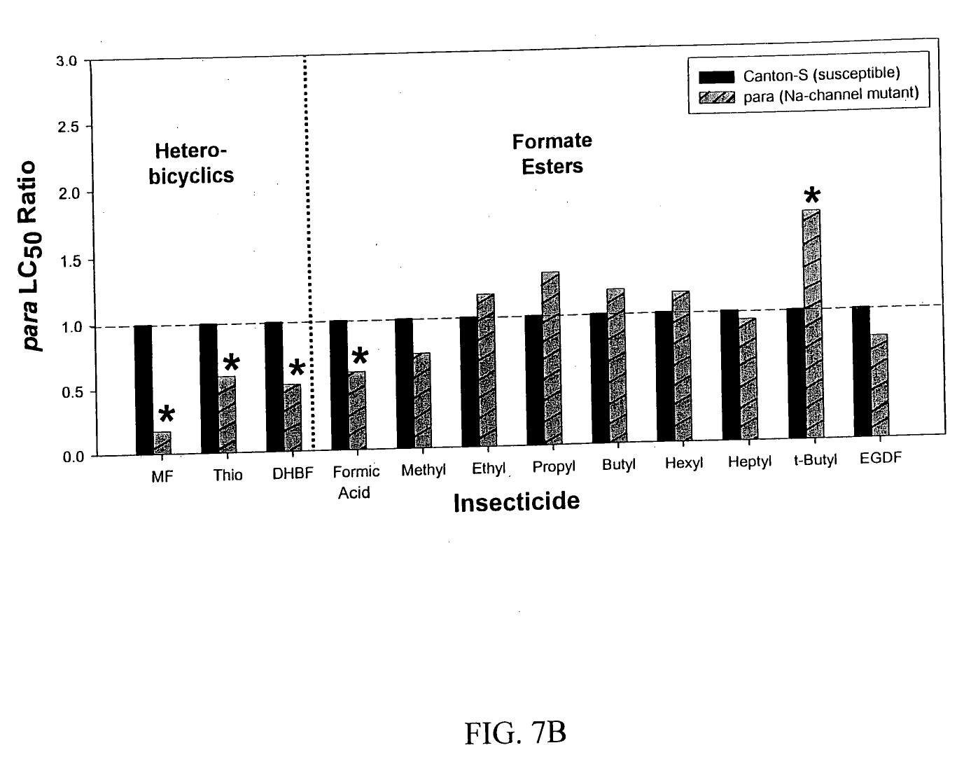

Two well-characterized neurological mutant strains were also tested. These strains include Rd1, which possesses a chloride channel point mutation, and para-ts1, which possesses a sodium channel point mutation. The responses of these two fly strains were compared to Canton-S in order to infer potential neurological effects for the various heterobicyclic and formate ester compounds (FIG. 7). Detailed probit analysis results for Rd1 and para-ts1 can be found in Table 2. Results for the heterobicyclics are as follows. First, significantly enhanced susceptibility was observed in Rd1 to both mentho- and benzothiophene. Second, para-ts1 showed enhanced susceptibility to mentho-, thio- and DHB-furan. Results for the formate esters and formic acid were markedly different. First, para-ts1 showed enhanced susceptibility to formic acid, but elevated tolerance to t-butyl formate. Second, Rd1 displayed significant resistance to formic acid, propyl formate and t-butyl formate. Third, non-significant tolerance was observed for both Rd1 and para-ts1 to several of the formate esters. These findings support the idea that compounds from both the heterobicyclic and formate ester groups are capable of eliciting broad-spectrum neurological impacts.

### EXAMPLE 8

### Metabolism

Heterobicyclic metabolism has received only limited attention in insects; however, cytochrome P450 is linked to heterobicyclic metabolism in both insects (Gunderson et al., 1986) and higher animals (Thomassen et al., 1991). In particular, P450-based aliphatic hydroxylation and ring hydroxylation seem to be very important in mammals (Thomassen et al., 1991), and can result in either detoxification or activation (Chen et al., 2003). With respect to activation, menthofuran and benzothiophene bioassays indicated greater susceptibility in Hikone-R than Canton-S (FIG. 5). These findings suggest that Hikone-R, which possesses elevated P450 levels, has a greater ability than Canton-S to convert mentho- and benzothiophene to toxic oxidative metabolites. PBO treatment, alternatively, resulted in increased menthofuran toxicity to Canton-S and no effects on Hikone (FIG. 6). Together, these results imply that some P450 isozymes lead to activation while others lead to detoxification. In other words, differential P450 isozyme expression profiles can apparently result in variable heterobicyclic toxicity. Evidence in support of this conclusion is the presence of 83 functional P450 genes in the Drosophila genome, all with potentially non-overlapping substrate specificities (Tijet et al., 2001; Feyereisen, 2005). Hikone-R over-expresses two P450 genes (Cyp6g1 and Cyp12d1; Le Goff et al., 2003; Festucci-Buselli et al., 2005); thus, our findings suggest that the Cyp6g1 and 12d1 proteins are the P450 isozymes responsible for heterobicyclic activation. By the same logic, our results suggest that either Cyp6g1 or 12d1 (or both) are responsible for DDVP detoxification and MITC activation, as well as EGDF detoxification and formate ester activation (see below). However, the presence of other non-P450 mechanisms in Hikone-R has not been well-investigated, thus, it is possible that other mechanisms may be acting in Hikone-R.

Because of the ester linkages contained in the formate esters, it is reasonable to expect that they should be acted upon by hydrolases to liberate the active metabolite formic acid, as well as potentially toxic aliphatic alcohols (Haritos and Dojchinov, 2003). Additionally, the formate esters have structures with a high probability of being acted upon by P450, including both alkyl chains and ester linkages (reviewed in Siegfried and Scharf, 2001). For these reasons, we tested the formate esters on the Hikone-R and Canton-S strains, both alone and in combination with DEF and PBO. Hikone-R is tolerant towards a number of formate esters, but because of atypical probit responses LC₅₀ ratios for only butyl formate and EGDF were significant (FIG. 5). Interestingly, Hikone also displayed greater susceptibility to formic acid than Canton-S. Because formic acid acts via cytochrome-C oxidase inhibition (Nicholls, 1975), it is possible that P450-connected redox machinery is linked to increased susceptibility by Hikone. Future research will be required to address this topic.

From synergist bioassays involving formate esters, PBO results suggest that all formate esters except methyl formate and EGDF are converted to more toxic metabolites by P450. Additionally, these findings further suggest that methyl formate and EGDF are detoxified by P450s other than Cyp6g1 and/or 12d1 (FIGS. 6A & 6C). DEF bioassays indicate an equally important role for esterases in activation of methyl, ethyl and butyl formate. In this respect, it is extremely noteworthy that the DEF synergism ratio for ethyl formate was >12-fold, which indicates a >12-fold reduction in ethyl formate toxicity after esterase inhibition. This finding is in good agreement with findings by Haritos and Dojchinov (2003) that implicated esterase-based liberation of formic acid as a major factor contributing to ethyl formate toxicity in the stored product pest Sitophilus oryzae. The current findings, particularly those relating to hydrolysis-based activation, provide rationale for further investigations into formate ester hydrolysis, cytochrome-C oxidase inhibition, and aliphatic alcohol toxicity.

### EXAMPLE 9

### Neurotoxicity

To examine for potential neurological effects of both volatile insecticide groups, we tested two well-characterized insecticide resistant neurological mutant strains. The GABA-gated chloride channel mutant strain Rd1 has a point mutation that confers cyclodiene and phenylpyrazole insecticide resistance. The sodium channel mutant strain para-ts1 has a point mutation that confers temperature-induced paralysis, insecticide resistance to DDT, and enhanced susceptibility to some pyrethroids. See Materials and Methods for detailed strain descriptions. The rationale for this approach is that, if resistance or increased susceptibility is observed in either Rd1 or para-ts1 for a given insecticide, this would indicate (respectively) neurological activity by that insecticide at either the GABA-gated chloride channel or sodium channel. Interestingly, both Rd1 and para-ts1 showed significantly enhanced susceptibility in the majority of heterobicyclic bioassays. The only bioassay in which Canton-S and Rd1 displayed identical toxicity responses was with DHBF. In agreement with these findings, Pedra et al. (2004) previously identified increased susceptibility by para-ts1 to the pyrethroid insecticide deltamethrin. The similarly enhanced susceptibilities of Rd1 and para-ts1 to the heterobicyclics are not readily explainable; further research will be necessary to better understand this phenomenon. However, one possible explanation is that the heterobicyclics have broad impacts across the nervous system, which are enhanced by modified chloride and sodium channel function. For example, some natural heterobicyclic-like compounds are known to elicit toxic effects by binding proteins indiscriminately (Zhou et al., 2004).

With respect to the formate esters, Rd1 showed significant resistance to propyl and t-butyl formate, and non-significant tolerance to butyl, hexyl and heptyl formate. Interestingly, Rd1 also has significant ˜2.5-fold resistance to formic acid, suggesting that formic acid has neurological activity at the GABA-gated chloride channel. Similar tolerance trends were observed for Rd1 across a broad range of formate esters; this supports synergist results as discussed above, and suggests that formate ester hydrolysis to formic acid is an activating metabolic step. Whether or not formic acid is also a respiratory disruptor in Drosophila via cytochrome-C oxidase inhibition (Nicholls, 1975; Petersen, 1977) also needs to be verified.

Also, with particular reference to t-butyl formate, it is noteworthy that both para-ts1 and Rd1 displayed significant tolerance to t-butyl formate. para-ts1 additionally showed non-significant tolerance to ethyl, propyl, butyl and hexyl formate. These findings not only link formate ester action to the sodium channel, but they also suggest that t-butyl formate has broad neurological impacts. Finally, the results for EGDF against both Rd1 and para-ts1 suggest that its toxicity is mediated by the parent compound EGDF, rather than some type active hydrolytic metabolite, i.e., formic acid. Overall, the neurological-mutant findings presented here show at least partial neurological modes of action for both heterobicyclics and formate esters.

### EXAMPLE 10

### Implications for Applied Vector Management

From these studies, a number of important trends emerged with respect to both pest management and resistance management. In relation to pest management, menthofuran currently shows the most promise in terms of being the most active/lowest rate material. Our findings for menthofuran are similar to those observed previously with pennyroyal oil, the crude source of menthofuran, in head lice (Yang et al., 2004). Other compounds, including benzothiophene, DHBF, and butyl, hexyl and heptyl formate also show effectiveness, and may offer greater safety through higher insect selectivity (Scharf et al., 2006). Additionally, EDGF has excellent knockdown characteristics that would offer distinct advantages for control of small-bodied dipteran pests of medical importance. Mixing menthofuran with the P450 inhibitor PBO significantly improved its efficacy to levels on the same scale (<100 μg/l) as the proven fumigant insecticides MITC and DDVP. Thus, use of P450 inhibitors is contemplated for enhancing heterobicyclic performance. As an alternative to conventional synergists, mixtures of any of the active insecticides with EGDF can provide for synergistically enhanced toxicity of both mixture components.

With respect to resistance management, several interesting trends were observed in relation to the concept of negative cross-resistance (NCR). As outlined by Pittendrigh and Gaffney (2001), NCR occurs “when a mutant allele confers (i) resistance to one toxic chemical and (ii) hyper-susceptibility to another”. Nine significant instances of NCR (and several other non-significant instances) were observed that are related to P450-based metabolism and target site mutations in both chloride and sodium channels. Regarding metabolism-based NCR, over-expression of the P450 genes Cyp6g1 and Cyp12d1 in the Hikone-R strain (LeGoff et al. 2003; Festucci-Buselli et al., 2005) confers resistance to malathion (Sundseth et al., 1989), DDT, neonicotinoids (Daborn et al., 2001) and DDVP (present study), but enhanced susceptibility to MITC, menthofuran and benzothiophene (FIG. 5). Thus, menthofuran and benzothiophene can have applications in managing insect populations resistant to other insecticide classes by P450-based metabolism. Also, cytochrome P450 apparently participates in the activation of a number of formate esters (FIGS. 6A & 6C), which after greater selection intensity, could eventually contribute to NCR with DDT, neonicotinoids and organophosphates such as malathion and DDVP. Regarding target site insensitivity, by the same thinking (FIG. 7), mentho-, thio-, and DHB-furan can all have uses in managing insect populations resistant to neurotoxins that are impacted by Rd1- and para-like mutations. Most importantly, because of their increased toxicity to metabolic and neurologically resistant strains, menthofuran and benzothiophene are apparently “generalized NCR toxins” (Pittendrigh and Gaffney, 2001) with broad potential for management of resistance to a diversity of insecticides.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

TABLE 2
Toxicity of volatile insecticides to insecticide-susceptible (S) and resistant (R) strains of
Drosophila melanogaster at 24 hr.

| | | | Slope ± Std. | Chi- | | |
|---|---|---|---|---|---|---|
| Compound | Strain | N | Error^{a} | Square^{b} | LC₅₀ (95% CL)^{c} | |
| Dichlorvos | Canton-S | 140 | 4.43 ± 0.36 | 0.25 | 11.1 | (9.6-13.0) |
| (DDVP) | Hikone-R | 220 | 4.64 ± 0.59 | 2.88 | 24.2 | (21.6-27.2) |
| Methylisothio- | Canton-S | 240 | 6.19 ± 1.07 | 1.62 | 95.2 | (83.7-108.6) |
| cyanate (MITC) | Hikone-R | 240 | 4.30 ± 0.62 | 1.07 | 62.4 | (52.9-73.1) |
| Menthofuran | Canton-S | 340 | 4.68 ± 0.42 | 7.65* | 411.4 | (375.2-447.4) |
| (MF) | Hikone-R | 240 | 4.12 ± 0.45 | 2.31 | 243.4 | (214.9-274.6) |
| | Rdl-R | 180 | 3.44 ± 0.46 | 0.34 | 260.7 | (223.0-310.9) |
| | para-R | 280 | 2.92 ± 0.50 | 7.42* | 72.0 | (42.9-105.0) |
| Benzothiophene | Canton-S | 200 | 5.37 ± 0.61 | 1.43 | 802.1 | (713.0-888.3) |
| (Thio) | Hikone-R | 230 | 4.42 ± 0.46 | 3.86 | 553.8 | (491.6-619.3) |
| | Rdl-R | 270 | 3.10 ± 0.54 | 6.68* | 204.2 | (105.7-298.0) |
| | para-R | 270 | 3.83 ± 1.02 | 23.10* | 473.9 | (158.5-1290.0) |
| Dihydro- | Canton-S | 250 | 4.50 ± 0.55 | 3.36 | 1479.0 | (1340.0-1669.0) |
| benzofuran | Hikone-R | 150 | 16.60 ± 6.02 | 5.92* | 1697.2 | (ND) |
| (DHBF) | Rdl-R | 150 | 6.87 ± 1.25 | 0.19 | 1496.0 | (1385.0-1640.0) |
| | para-R | 150 | 7.21 ± 1.50 | 2.39 | 764.1 | (651.9-836.2) |
| Formic Acid | Canton-S | 200 | 2.67 ± 1.47 | 0.78 | 7074.0 | (ND) |
| | Hikone-R | 400 | 3.29 ± 1.02 | 0.70 | 4660.0 | (3070.0->10000) |
| | Rdl-R | 200 | 1.16 ± 0.62 | 0.65 | 17163.0 | (ND) |
| | para-R | 200 | 2.00 ± 0.61 | 0.53 | 4215.0 | (2626.0->10000) |
| Methyl Formate | Canton-S | 120 | 3.27 ± 0.95 | 1.09 | 2471.0 | (1915.0-5336.0) |
| | Hikone-R | 400 | 2.72 ± 0.68 | 2.75 | 4474.0 | (3058.0-12000) |
| | Rdl-R | 200 | 9.41 ± 2.14 | 0.10 | 2070.0 | (1921.0-2395.0) |
| | para-R | 200 | 8.84 ± 1.46 | 0.49 | 1792.0 | (1678.0-1944.0) |
| Ethyl Formate | Canton-S | 120 | 5.18 ± 1.07 | 1.03 | 1656.0 | (1486.0-1917.0) |
| | Hikone-R | 200 | 12.50 ± 7.10 | 25.65 | 1791.0 | (ND) |
| | Rdl-R | 200 | 6.38 ± 1.91 | 8.53* | 1478.0 | (ND) |
| | para-R | 200 | 7.11 ± 1.34 | 0.01 | 1933.0 | (1778.0-2202.0) |
| Propyl Formate | Canton-S | 150 | 23.24 ± 3.22 | 1.92 | 1833.0 | (1787.0-1884.0) |
| | Hikone-R | 200 | 7.43 ± 1.26 | 0.58 | 1788.0 | (1660.0-1971.0) |
| | Rdl-R | 200 | 7.98 ± 2.36) | 5.81* | 1886.0 | (ND) |
| | para-R | 200 | 7.77 ± 2.36 | 0.11 | 2411.0 | (2117.0-3755.0) |
| Butyl Formate | Canton-S | 230 | 5.43 ± 0.62 | 1.97 | 913.1 | (820.7-996.4) |
| | Hikone-R | 190 | 8.82 ± 2.00 | 4.55 | 1570.0 | (1149.0->3000) |
| | Rdl-R | 200 | 7.28 ± 4.58 | 50.14* | 1246.0 | (ND) |
| | para-R | 200 | 5.17 ± 0.60 | 4.55 | 987.1 | (886.9-1085.0) |
| t-Butyl Formate | Canton-S | 120 | 22.21 ± 3.57 | 2.72 | 1981.0 | (1917.0-2048.4) |
| | Hikone-R | 400 | 44.70 ± ND | 0.00 | 2165.0 | (ND) |
| | Rdl-R | 200 | 3.76 ± 2.22 | 0.71 | 4946.0 | (ND) |
| | para-R | 200 | 3.91 ± 1.36 | 0.04 | 3422.0 | (2469.0->10,000) |
| Hexyl Formate | Canton-S | 200 | 9.23 ± 1.34 | 1.56 | 1140.0 | (1070.0-1200.0) |
| | Hikone-R | 200 | 3.26 ± 0.65 | 3.50 | 1541.0 | (1354.0-1833.0) |
| | Rdl-R | 200 | 8.19 ± 1.04 | 1.82 | 1511.0 | (1420.0-1610.0) |
| | para-R | 200 | 5.23 ± 1.15 | 5.05* | 1307.0 | (405.8-2714.0) |
| Heptyl Formate | Canton-S | 250 | 8.54 ± 1.21 | 1.33 | 1357.0 | (1267.0-1426.0) |
| | Hikone-R | 200 | 6.68 ± 0.95 | 3.74 | 1483.0 | (1378.0-1585.0) |
| | Rdl-R | 150 | 13.98 ± 2.33 | 0.32 | 1656.6 | (1584.2-1718.3) |
| | para-R | 200 | 9.25 ± 1.21 | 0.16 | 1257.0 | (1189.0-1321.0) |
| EGDF | Canton-S | 120 | 3.27 ± 0.95 | 0.77 | 2471.0 | (1915.0-5336.0) |
| (Ethylene Glycol | Hikone-R | 200 | 5.02 ± 2.41 | 0.32 | 3540.0 | (2474->5000) |
| Di-Formate) | Rdl-R | 200 | 5.50 ± 1.50 | 0.58 | 2536.0 | (2143.0-4101.0) |
| | para-R | 200 | 7.67 ± 1.43 | 0.28 | 1930.0 | (1785.0-2176.0) |

All Canton-S data are taken from Scharf et al. (2006). Experimental insecticides are from heterobicyclic and formate ester groups. Shown in bold are the positive control/standard insecticides DDVP, MITC and formic acid. The Hikone-R strain possesses resistance via elevated detoxification capabilities, Rdl via a point mutation in the GABA-gated chloride channel, and para-ts1 via a point mutation in the voltage-gated sodium channel.
^{a}Slope of the probit mortality line.
^{b}Pearson's Chi-square goodness-of-fit test, testing whether the data fit an expected concentration-mortality probit model. Values followed by “*” indicate a lack of fit relative to an expected concentration-mortality probit curve.
^{c}LC₅₀ values and 95% confidence limits (CL) are expressed in μg insecticide per 0.5 liter of headspace. “ND” indicates that confidence limits were not determinable due to lack of fit by raw data to the probit model.

TABLE 3
Effects of detoxification enzyme inhibitors on volatile insecticide toxicity at 24 hr.

| | | | Slope ± Std. | Chi- | | |
|---|---|---|---|---|---|---|
| Strain | Treatment | N | Error^{a} | Square^{b} | LC₅₀ (95% CL)^{c} | |
| Canton-S | Menthofuran | 340 | 4.68 ± 0.42 | 7.65 | 411.4 | (375.2-447.4) |
| | +PBO | 300 | 1.31 ± 0.21 | 1.40 | 233.4 | (172.7-352.2) |
| Hikone-R | Menthofuran | 240 | 4.12 ± 0.45 | 2.31 | 243.4 | (214.9-274.6) |
| | +PBO | 250 | 4.03 ± 1.18 | 23.60* | 241.3 | (66.0-1762.0) |
| Canton-S | Benzothiophene | 200 | 5.37 ± 0.61 | 1.43 | 802.1 | (713.0-888.3) |
| | +PBO | 280 | 6.76 ± 0.87 | 0.14 | 807.2 | (755.9-868.2) |
| Hikone-R | Benzothiophene | 230 | 4.42 ± 0.46 | 3.86 | 553.8 | (491.6-619.3) |
| | +PBO | 200 | 5.62 ± 0.69 | 1.65 | 576.5 | (521.5-630.7) |
| Canton-S | DHBF | 250 | 4.50 ± 0.55 | 3.36 | 1479.0 | (1340.0-1669.0) |
| | +PBO | 200 | 16.10 ± 2.30 | 4.40 | 1338.5 | (1280.9-1386.5) |
| Hikone-R | DHBF | 150 | 16.60 ± 6.02 | 5.92* | 1697.2 | (ND) |
| | +PBO | 200 | 10.70 ± 1.50 | 1.71 | 1620.0 | (1541.0-1695.0) |
| Canton-S | Methyl Formate | 120 | 3.27 ± 0.95 | 1.09 | 2471.0 | (1915.0-5336.0) |
| | +PBO | 200 | 49.50 ± ND | 0.00* | 2079.0 | (ND) |
| | +DEF | 200 | 3.98 ± 1.52 | 0.71 | 3675.0 | (2548.0->10000) |
| Hikone-R | Methyl Formate | 400 | 2.72 ± 0.68 | 2.75 | 4474.0 | (3058->10000) |
| | +PBO | 200 | 51.10 ± ND | 0.00* | 2058.8 | (ND) |
| | +DEF | 400 | 1.91 ± 0.60 | 0.01 | 8187.0 | (4099->10000) |
| Canton-S | Ethyl Formate | 120 | 5.18 ± 1.07 | 1.03 | 1656.0 | (1486.0-1917.0) |
| | +PBO | 200 | 4.74 ± 1.44 | 0.19 | 2856.0 | (2274.0-6436.0) |
| | +DEF | 200 | 1.73 ± 1.35 | 0.47 | 20500.0 | (ND) |
| Hikone-R | Ethyl Formate | 200 | 12.50 ± 7.10 | 25.56* | 1791.0 | (ND) |
| | +PBO | 200 | 5.17 ± 1.28 | 2.35 | 2451.0 | (2086.0-3667.0) |
| | +DEF | 200 | 14.17 ± 1.80 | 66.19* | 1742.0 | (ND) |
| Canton-S | Propyl Formate | 150 | 23.24 ± 3.22 | 1.92 | 1833.0 | (1787.0-1884.0) |
| | +PBO | 200 | 6.61 ± 1.63) | 2.23 | 2330.0 | (2053.0-3164.0) |
| | +DEF | 200 | 7.40 ± 1.76 | 0.62 | 2171.0 | (1964.0-2707.0) |
| Hikone-R | Propyl Formate | 200 | 7.43 ± 1.26 | 0.58 | 1788.0 | (1660.0-1971.0) |
| | +PBO | 200 | 6.41 ± 1.87 | 0.59 | 2482.0 | (2131.0-4042.0) |
| | +DEF | 200 | 6.15 ± 1.40 | 0.06 | 2213.0 | (1965.0-2850.0) |
| Canton-S | Butyl Formate | 230 | 5.43 ± 0.62 | 1.97 | 913.1 | (820.7-996.4) |
| | +PBO | 150 | 9.35 ± 1.28 | 0.77 | 1353.0 | (1268.0-1434.0) |
| | +DEF | 190 | 12.45 ± 2.27 | 0.50 | 1814.0 | (1740.0-1913.0) |
| Hikone-R | Butyl Formate | 190 | 8.82 ± 2.00 | 4.55 | 1570.0 | (1149.0->3000) |
| | +PBO | 200 | 9.08 ± 1.21 | 1.43 | 1703.0 | (1610.0-1827.0) |
| | +DEF | 140 | 13.48 ± 2.53 | 1.90 | 1853.0 | (1781.0-1958.0) |
| Canton-S | Hexyl Formate | 200 | 9.23 ± 1.34 | 1.56 | 1140.0 | (1070.0-1200.0) |
| | +PBO | 200 | 8.20 ± 0.94 | 2.41 | 1334.0 | (1250.0-1419.0) |
| | +DEF | 150 | 5.66 ± 1.81 | 3.59 | 1494.0 | (ND) |
| Hikone-R | Hexyl Formate | 200 | 3.26 ± 0.65 | 3.50 | 1541.0 | (1354.0-1833.0) |
| | +PBO | 200 | 6.51 ± 0.88 | 0.60 | 1552.0 | (1443.0-1681.0) |
| | +DEF | 150 | 7.62 ± 1.24 | 0.55 | 1766.0 | (1642.0-1937.0) |
| Canton-S | Heptyl Formate | 250 | 8.54 ± 1.21 | 1.33 | 1357.0 | (1267.0-1426.0) |
| | +PBO | 200 | 7.90 ± 1.60 | 5.12 | 1497.0 | (749.9-2080.0) |
| | +DEF | 150 | 3.32 ± 0.62 | 0.64 | 1185.0 | (1017.0-1458.0) |
| Hikone-R | Heptyl Formate | 200 | 6.51 ± 0.88 | 3.74 | 1483.0 | (1378.0-1585.0) |
| | +PBO | 200 | 13.04 ± 2.15 | 0.34 | 1758.0 | (1690.0-1833.0) |
| | +DEF | 150 | 7.41 ± 1.06 | 2.08 | 1505.0 | (1397.0-1623.0) |
| Canton-S | tert-Butyl-Formate | 120 | 22.21 ± 3.57 | 2.72 | 1981.0 | (1917.0-2048.4) |
| | +PBO | 200 | 5.79 ± 3.24 | 0.06 | 3476.0 | (ND) |
| | +DEF | 200 | 43.42 ± ND | 0.00 | 2194.6 | (ND) |
| Hikone-R | tert-Butyl-Formate | 400 | 44.70 ± ND | 0.00 | 2165.4 | (ND) |
| | +PBO | 200 | 42.11 ± ND | 0.00 | 2237.7 | (ND) |
| | +DEF | 150 | 44.30 ± ND | 0.00 | 2178.0 | (ND) |
| Canton-S | EGDF | 120 | 3.27 ± 0.95 | 0.77 | 2471.0 | (1915.0-5336.0) |
| | +PBO | 150 | 4.21 ± 0.12 | 0.24 | 2716.0 | (2179.0-5649.0) |
| | +DEF | 200 | 2.31 ± 0.46 | 2.05 | 1696.0 | (1397.0-2319.0) |
| Hikone-R | EGDF | 200 | 5.02 ± 2.41 | 0.32 | 3540.0 | (2474.0->5000) |
| | +PBO | 150 | 6.81 ± 1.53 | 1.03 | 2183.0 | (1961.0-2328.0) |
| | +DEF | 150 | 4.33 ± 1.38 | 0.09 | 2878.0 | (2259.0-7405.0) |

Drosophila strains tested were the insecticide-susceptible Canton-S strain and the metabolically resistant Hikone-R strain. Synergists tested were piperonyl butoxide (PBO) and sss-tributyl-phosphorotrithioate (DEF), both delivered at 10 μg per bioassay vial. All non-synergist Canton-S data are taken from Scharf et al. (2006).
^{a}Slope of the best-fit probit mortality line.
^{b}Pearson's Chi-square goodness-of-fit test., testing whether the data fit an expected concentration-mortality probit model. Values followed by “*” indicate a lack of fat relative to an expected concentration-mortality probit model.
^{c}LC values and 95% confidence limits (CL) are expressed in μg insecticide per 0.5 liter of headspace. “ND” indicates that confidence limits were not determinable due to lack of fit by raw data to probit model (i.e., Chi-square >5.0).

### REFERENCES

Bloomquist, J. R., R. H. ffrench-Constant, and R. T. Roush (1991) Excitation of central neurons by dieldrin and picrotoxinin in insecticide susceptible and resistant Drosophila melanogaster. Pestic. Sci. 32: 463-469.
Bloomquist, J. R., R. T. Roush, and R. H. ffrench-Constant (1992) Reduced neuronal sensitivity to dieldrin and picrotoxinin in a cyclodiene-resistant strain of Drosophila melanogaster. Arch. Insect Biochem. Physiol. 19: 17-25.
Bloomquist, J. R. (2000) GABA and glutamate receptors as biochemical sites for insecticide action, pp. 17-42. In Ishaaya, I. (ed.), Biochemical sites of insecticide action and resistance. Springer-Verlag, New York.
Brown A. W. A. (1951) Insect control by chemicals, Chapman and Hall, New York.
Bell C H. (2000) “Fumigation in the 21st century,” Crop Protect. 19: 563-539.
Caddick L. (2004) “Search for methyl bromide and phosphine alternatives,” Outlooks Pest Manage. June: 118-119.
Chen L. J., Lebetkin E. H., Burka L. T. (2003) “Metabolism of menthofuran in Fischer-344 rats: identification of sulfonic acid metabolites,” Drug Metab. Dispos. 31: 1208-1213.
Daborn, P. J., S. Boundy, J. J. Yen, B. R. Pittendrigh, and R. H. ffrench-Constant (2001) DDT resistance in Drosophila correlates with Cyp6g1 over-expression and confers cross-resistance to the neonicotinoid imidacloprid. Mol. Gen. Genet. 266: 556-563.
Dyreborg S., Arvin E. and Broholm K., (1996) “Effects of creosote compounds on the aerobic bio-degradation of benzene,” Biodegradation 7: 191-201.
Ferguson J. and Pirie H. (1948) “The toxicity of vapours to the grain weevil,” Ann. Appl. Biol. 35: 532-550.
Festucci-Buselli, R. A., A. S. Carvalho-Dias, M. de Oliviera-Andrade, C. Caixeta-Nunes, H. M. Li, J. J. Stuart, W. M. Muir, M. E. Scharf, and B. R. Pittendrigh (2005) Expression of Cyp6g1 and Cyp12d1 in DDT resistant and susceptible strains of Drosophila melanogaster. Insect Mol. Biol. 14: 69-78.
ffrench-Constant, R. H., R. T. Roush, D. Mortlock, and G. Dively (1990) Isolation of dieldrin resistance from field populations of Drosophila melanogaster. J. Econ. Entomol. 83: 1733-1737.
ffrench-Constant, R. H. and R. T. Roush (1991) Gene mapping and cross-resistance in cyclodiene insecticide-resistant Drosophila melanogaster. Genet. Res. 57: 17-21.
ffrench-Constant, R. H., T. A. Rocheleau, J. C. Steichen, and A. E. Chalmers (1993) A point mutation in a Drosophila GABA receptor confers insecticide resistance. Nature 363: 449-451.
ffrench-Constant R. H., Daborn P. J. and Le Goff G. (2004) “The genetics and genomics of insecticide resistance,” Trends Genet. 20: 163-170.
ffrench-Constant R. H., Rocheleau T. A., Steichen J. C., and Chalmers A. E. (1993) “A point mutation in a Drosophila GABA receptor confers insecticide resistance,” Nature 363: 451-453.
Feyereisen, R (2005) Insect cytochrome P450, pp. 1-77. In L. I. Gilbert, K. Iatrou, and S. S. Gill [eds.], Comprehensive Molecular Insect Science [vol. 4]: Biochemistry and Molecular Biology. Elsevier, Amsterdam.
Gunderson C. A., Brattsten L. B., and Fleming J. T. (1986) “Microsomal oxidase and glutathione transferase as factors influencing the effects of pulegone in southern and fall armyworm,” Pestic. Biochem. Physiol. 26: 238-249.
Haritos V. S. and Dojchinov G. (2003) “Cytochrome c oxidase inhibition in the rice weevil Sitophilus oryzae by formate, the toxic metabolite of alkyl formats,” Comp. Biochem. Physiol. C 136: 135-143.
Haritos, V. S., K. A. Damcevski, and G. Dojchinov (2006) Improved efficacy of ethyl formate against stored grain insects by combination with carbon dioxide in a ‘dynamic’ application. Pest Manage. Sci. 62: 325-333.
Jang, Y. S., Y. C. Yang, D. S. Choi, and Y. J. Ahn (2005) Vapor phase toxicity of marjoram oil compounds and their related monoterpenoids to Blattella germanica. J. Agric. Food Chem. 53: 7892-7898.
Kim, S. I., H. K. Kim, Y. Y. Koh, J. M. Clark, and Y. J. Ahn (2006) Toxicity of spray and fumigant products containing cassia oil to Dermatophagoides farinae and Dermatophagoides pteronyssinus. Pest Manage. Sci. 62: 768-774.
Lang, E., and H. Lang (1972) Spezifische farbreaktion zum direkten nachweis der ameisensäure. Z. Anal. Chem. 260: 8-10.
Le Goff, G., S. Boundy, P. J. Daborn, J. J. Yen, L. Sofer, R. Lind, C. Sabourault, L. Madi-Ravazzi, and R. H. ffrench-Constant (2003) Microarray analysis of cytochrome P450 mediated insecticide resistance in Drosophila. Insect Biochem. Molec. Biol. 33: 701-708.
Loughney, K., R. Kreber, and B. Ganetzky (1989) Molecular analysis of the para locus, a sodium channel gene in Drosophila. Cell 58: 1143-1154.
Lush I. E. and Andrews K. M. (1978) “Genetic variation between mice in their metabolism of coumarin and its derivatives,” Genet. Res. 31: 177-186.
Mallis A. (1954) Handbook of pest control, 2nd Ed., Gulf Research and Development Co., Pittsburg.
Martin R. L., Pittendrigh B. R., Reenan R., ffrench-Constant R. H., and Hanck D. A. (2000) “Point mutations in domain III of a Drosophila neuronal Na channel confer resistance to allethrin,” Insect Biochem. Molec. Biol. 30: 105-109.
Nicholls P. (1975) “Formate as an inhibitor of cytochrome c oxidase,” Biochem. Biophys. Res. Comm. 67: 610-616.
Park I. K. and Shin S. C. (2005) “Fumigant activity of plant essential oils and components from garlic (Allium sativum) and clove bud (Eugenia caryophyllata) oils against the Japanese termite Reticulitermes speratus,” J. Agric. Food Chem. 53: 4388-4392.
Park, I. K., K. K. Choi, D. H. Kim, I. H. Choi, L. S. Kim, W. C. Bak, J. W. Choi, and S. C. Shin (2006). Fumigant activity of plant essential oils and components from horseradish, anise and garlic oils against Lycoriella ingenua. Pest Manage. Sci. 62: 723-728.
Pedra, J. H. F., A. Hostetler, P. J. Gaffney, R. A. Reenan, and B. R. Pittendrigh (2004) Hyper-susceptibility to deltamethrin in para^{ts-1} DDT resistant Drosophila melanogaster. Pestic. Biochem. Physiol. 78: 58-66.
Pedra J. H. F., McIntyre L. M., Scharf M. E., and Pittendrigh B. R. (2004) “Genome-wide transcription profile of field- and laboratory-selected DDT-resistant Drosophila,” Proc. Nat. Acad. Sci. USA. 101: 7034-7039.
Petersen, L. C. (1977) The effect of inhibitors on the oxygen kinetics of cytochrome c oxidase. Biochem. Biophys. Acta 460: 299-307.
Pittendrigh, B. R., R. Reenan, R. H. ffrench-Constant, and B. Ganetzky (1997) Point mutations in the Drosophila sodium channel gene para associated with resistance to DDT and pyrethroid insecticides. Mol. Gen. Genet. 256: 602-610.
Pittendrigh, B. R. and P. J. Gaffney (2001) Pesticide resistance: can we make it a renewable resource? J. Theor. Biol. 211: 365-375.
Pratt S. J. and Reuss R. (2004) “Scrubbing carbon dioxide prevents overestimation of insect mortality in long-duration static phosphine toxicity assays,” J. Stored Prod. Res. 40: 233-239.
Robertson, J. R. and H. K. Preisler (1992) Pesticide bioassays with arthropods. CRC Press, Boca Raton.
Salgado V. L. (1997) “The mode of action of spinosad and other insect control products,” Down to Earth News 52: 35-43.
Scharf M. E. and Siegfried B. D. (1999) “Toxicity and neurophysiological effects of fipronil and fipronil-sulfone on the western corn rootworm,” Arch. Insect Biochem. Physiol. 40: 150-156.
Scharf M. E., Bennett G. W., Reid B. L., and Qiu C. (1995) “A comparison of three insecticide resistance detection methods for the German cockroach,” J. Econ. Entomol. 88: 536-542.
Scharf M. E., Meinke L. J., Siegfried B. D., Wright R. J., and Chandler L. D. (1999) “Carbaryl susceptibility, diagnostic concentration determination and synergism for U.S. populations of western corn rootworm,” J. Econ. Entomol. 92: 33-39.
Scharf, M. E., S. N. Nguyen, and C. Song (2006) Evaluation of volatile low molecular weight insecticides using Drosophila melanogaster as a model. Pest Manage. Sci. 62: 655-633.
Siegfried, B. D. and M. E. Scharf (2001) Mechanisms of organophosphate resistance in insects, pp. 269-291. In I. Ishaaya [ed.], Biochemical sites of insecticide action and resistance. Springer-Verlag, Berlin.
Sim, M. J., D. R. Choi, and Y. J. Ahn (2006) Vapor phase toxicity of plant essential oils to Cadra cautella. J. Econ. Entomol. 99: 593-598.
Sundseth, S. S., S. J. Kennel, and L. C. Waters (1989) Monoclonal antibodies to resistance-related forms of cytochrome P450 in Drosophila melanogaster. Pestic. Biochem. Physiol. 33: 176-188.
Suzuki, D. T., T. Grigliatti, and R. Williamson (1971) Temperature-sensitive mutations in Drosophila melanogaster, VII. A mutation (para^{ts}) causing reversible adult paralysis. Proc. Nat. Acad. Sci. USA 68: 890-893.
Tattersfield F. and Roberts A. W. R. (1920) “Toxicity and boiling point of fumigants,” Ann. Appl. Biol. 10: 199-232.
Thomassen, D., N. Knebel, J. T. Slattery, R. H. McClanahan, and S. D. Nelson (1991) Reactive intermediates in the oxidation of menthofuran by cytochromes P-450. Chem. Res. Toxicol. 5: 123-130.
Tijet, N., C. Helvig, and R. Feyereisen (2001) The cytochrome P450 gene superfamily in Drosophila melanogaster: annotation, intron-exon organization and phylogeny. Gene 262: 189-198.
Waters, L. C., S. I. Simms, and C. E. Nix (1984) Natural variation in the expression of cytochrome P-450 and dimethylnitrosamine demethylase in Drosophila. Biochem. Biophys. Res. Comm. 123: 907-913.
Yang, Y. C., H. S. Lee, J. M. Clark, and Y. J. Ahn (2004) Insecticidal activity of plant essential oils against Pediculus humanus capitis. J. Med. Entomol. 41: 699-704.
Zhou S. F., Koh H. L., Gao Y. H., Gong Z. Y., and Lee J. D. (2004) “Herbal bioactivation: the good, the bad and the ugly,” Life Sci. 74: 935-968.

We claim:

1. A bioassay for screening volatile compounds for activity to kill a pest, said method comprising:
a) providing one or more pests in a first container that permits gas exchange;
b) providing said first container within a second container that comprises a liquid absorbent material, wherein said liquid absorbent material is absorbed with a compound or mixture of compounds to be screened for activity;
c) sealing said second container wherein said one or more pests are exposed to said compound or mixture of compounds; and
d) determining the mortality of said one or more pests exposed to said compound or mixture of compounds.

2. The bioassay according to claim 1, wherein said pest is an insect.

3. The bioassay according to claim 2, wherein said one or more insect is a fly.

4. The bioassay according to claim 3, wherein said fly is a Drosophila species.

5. The bioassay according to claim 1, wherein said compound or mixture of compounds is provided in solution at a concentration of between about 10 μg/μl to about 1000 μg/μl.

6. The bioassay according to claim 1, wherein said compound or mixture of compounds is provided in a solvent selected from the group consisting of acetone, ethanol, methanol, methyl cellosolve, dimethyl sulfoxide (DMSO), and hexane.

7. The bioassay according to claim 1, wherein said one or more pests are exposed to said compound or mixture of compounds for between about 12 hours to about 48 hours.

8. The bioassay according to claim 1, wherein a food substance is provided within said first container.

9. The bioassay according to claim 1, wherein an inhibitor of a cytochrome P450 enzyme is also provided in said second container.

10. The bioassay according to claim 9, wherein said inhibitor is piperonyl butoxide (PBO).

11. The bioassay according to claim 1, wherein an inhibitor of an esterase enzyme is also provided in said second container.

12. The bioassay according to claim 11, wherein said inhibitor is SSS-tributyl-phosphorotrithioate (DEF).

13. A method for killing a pest, said method comprising exposing or contacting a pest with an effective amount of a volatile compound identified using a method according to claim 1.

14. The method according to claim 13, wherein said pest is an insect.

15. The method according to claim 14, wherein said insect is a fly.

16. The method according to claim 15, wherein said fly is a Drosophila species.

17. The method according to claim 13, wherein said volatile compound is formulated as a fumigant.

18. The method according to claim 13, wherein said volatile compound is in undiluted form.

19. The method according to claim 13, wherein said volatile compound is mixed with or provided with an inert gas.

20. The method according to claim 13, wherein said volatile compound is provided in liquid form.

21. The method according to claim 13, wherein said insect is Aedes spp., Anopheles spp., Culex spp. (including Culex nigripalpus), Drosophila melanogaster, Musca spp. (including Musca domestica), Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio spp. (including Bibio hortulanus), Oscinella frit, Phorbia spp., Pegomyia hyoscyami, Ceratitus capitata, Dacus oleae, or Tipula paludosa.

22. The method according to claim 13, wherein said compound is menthofuran, benzothiophene, dihydrobenzofuran, coumaran, 9,9-difluoro-4-methyl-7-oxabicyclo[4.3.0]non-3-ene, 4-methyl-7-oxabicyclo[4.3.0]non-1(6),3-diene, dimethyl-coumarone, indole, formic acid, methyl formate, ethyl formate, propyl formate, butyl formate, hexyl formate, heptyl formate, t-butyl formate, ethylene glycol di-formate, 1,2-propylene glycol diformate, 1,3-propylene glycol diformate, 1,4-propylene glycol diformate, cyclopentyl formate, methyl acetate, ethyl acetate, propyl acetate, n-butyl acetate, pentyl acetate, hexyl acetate, iso-propyl acetate, t-butyl acetate, methyl propionate, ethyl propionate, propyl propionate, butyl propionate, methyl butyrate, ethyl butyrate, propyl butyrate, methyl valerate, or ethyl valerate, or any combination of said compounds.

23. The method according to claim 13, wherein said volatile compound is formulated with an inhibitor of a cytochrome P450 enzyme.

24. The method according to claim 23, wherein said inhibitor is piperonyl butoxide (PBO).

25. The method according to claim 13, wherein said volatile compound is formulated with an inhibitor of an esterase enzyme.

26. The method according to claim 25, wherein said inhibitor is SSS-tributyl-phosphorotrithioate (DEF).

27. A pesticidal formulation, wherein said formulation comprises a volatile compound identified using a method according to claim 1.

28. The pesticidal formulation according to claim 27, wherein said compound is menthofuran, benzothiophene, dihydrobenzofuran, coumaran, 9,9-difluoro-4-methyl-7-oxabicyclo[4.3.0]non-3-ene, 4-methyl-7-oxabicyclo[4.3.0]non-1(6),3-diene, dimethyl-coumarone, indole, formic acid, methyl formate, ethyl formate, propyl formate, butyl formate, hexyl formate, heptyl formate, t-butyl formate, ethylene glycol di-formate, 1,2-propylene glycol diformate, 1,3-propylene glycol diformate, 1,4-propylene glycol diformate, cyclopentyl formate, methyl acetate, ethyl acetate, propyl acetate, n-butyl acetate, pentyl acetate, hexyl acetate, iso-propyl acetate, t-butyl acetate, methyl propionate, ethyl propionate, propyl propionate, butyl propionate, methyl butyrate, ethyl butyrate, propyl butyrate, methyl valerate, or ethyl valerate, or any combination of said compounds.

29. The pesticidal formulation according to claim 27, wherein said formulation comprises an inhibitor of a cytochrome P450 enzyme.

30. The pesticidal formulation according to claim 29, wherein said inhibitor is piperonyl butoxide (PBO).

31. The pesticidal formulation according to claim 27, wherein said formulation comprises an inhibitor of an esterase enzyme.

32. The pesticidal formulation according to claim 31, wherein said inhibitor is SSS-tributyl-phosphorotrithioate (DEF).

33. The pesticidal formulation according to claim 27, wherein said pesticidal formulation is formulated as a fumigant.

34. The pesticidal formulation according to claim 27, wherein said volatile compound is in undiluted form.

35. The pesticidal formulation according to claim 27, wherein said volatile compound is mixed with or provided with an inert gas.

36. The pesticidal formulation according to claim 27, wherein said volatile compound is provided in liquid form.

37. An apparatus for conducting a bioassay for screening volatile compounds for activity against pests, comprising:
a) a first container for containing one or more pests, wherein said first container permits gas exchange; and
b) a second container that can contain said first container and that can contain a liquid absorbent material, wherein said second container is releasably sealable.

38. The apparatus according to claim 37, wherein said first container comprises a food substance for said one or more pests.

39. The apparatus according to claim 37, wherein said liquid absorbent material is absorbed with a test compound.

40. The apparatus according to claim 39, wherein said test compound is provided in a solvent that exhibits little or no toxicity to said one or more pests.

41. The apparatus according to claim 40, wherein said solvent is acetone, ethanol, methanol, methyl cellosolve, DMSO, or hexane.

42. The apparatus according to claim 37, wherein said first container has at least one open end comprising a material that prevents said one or more pests from escaping said first container and permits gas exchange with said second container.
